(19) United States
(12) Patent Application Publication
(10) Pub. No.: US 2024/0316052 A1
(43) Pub. Date: Sep. 26, 2024

(54) MITOCHONDRIAL SUPERCOMPLEX FORMATION PROMOTER, COMPOSITION FOR MAINTAINING OR IMPROVING MUSCULAR STRENGTH, THERAPEUTIC OR PREVENTATIVE PHARMACEUTICAL COMPOSITION FOR DISEASES WHICH DECREASE MITOCHONDRIAL FUNCTION OR MUSCULAR FUNCTION, AND SCREENING METHOD FOR SUBSTANCES WHICH CONTRIBUTE TO FORMATION OF MITOCHONDRIAL SUPERCOMPLEX

(71) Applicant: TOKYO METROPOLITAN GERIATRIC HOSPITAL AND INSTITUTE OF GERONTOLOGY, Tokyo (JP)

(72) Inventors: Satoshi INOUE, Tokyo (JP); Kotaro AZUMA, Tokyo (JP); Ami KOBAYASHI, Tokyo (JP)

(73) Assignee: TOKYO METROPOLITAN GERIATRIC HOSPITAL AND INSTITUTE OF GERONTOLOGY, Tokyo (JP)

(21) Appl. No.: 18/564,253

(22) PCT Filed: May 31, 2022
(86) PCT No.: PCT/JP2022/022074

§ 371 Date: Jun. 11, 2024

(30) Foreign Application Priority Data
May 31, 2021 (JP) 2021-091600

(51) Int. Cl. A61K 31/519; A61K 31/506; A61P 3/00; A61P 21/00; C12N 15/113; C12N 15/85; G01N 33/50

(52) U.S. Cl. CPC A61K 31/519; A61K 31/506; A61P 3/00; A61P 21/00; C12N 15/1137; C12N 15/85; G01N 33/5023; C12N 2310/14; C12N 2503/02; C12N 2800/107

(57) ABSTRACT

The object of the present invention is to provide a method for treating or preventing diseases related to mitochondrial function by a substance related to the improvement or decrease of mitochondrial function. The above problem can be solved by the agent for promoting formation of mitochondrial respiratory chain supercomplex, comprising Syk inhibitor as an active ingredient. a composition for maintaining or enhancing muscle strength, comprising the agent for promoting formation of mitochondrial respiratory chain supercomplex, or the pharmaceutical composition for treating or preventing muscle hypofunction disease or mitochondrial hypofunction disease, comprising the agent for promoting formation of mitochondrial respiratory chain supercomplex, of the present invention.

### CROSS REFERENCE TO RELATED APPLICATIONS

This application is a U.S. National Phase filing under 35 U.S.C. § 371 of International Application PCT/JP2022/022074, filed May 31, 2022, and published as WO 2022/255346 A1 on Dec. 8, 2022. PCT/JP2022/022074 claims priority from Japanese application number 2021-091600, filed May 31, 2021. The entire contents of each of these prior applications are hereby incorporated herein by reference.

### SEQUENCE LISTING

The instant application contains an electronic sequence listing. The contents of the electronic sequence listing (ASCII; H2852033.txt; Size: 16,684 bytes; and Date of Creation: May 31, 2022) is herein incorporated by reference in its entirety.

### DESCRIPTION

### Technical Field

The present invention relates to an agent for promoting formation of mitochondrial respiratory chain supercomplex, a composition for maintaining or enhancing muscle strength, and a pharmaceutical composition for treating or preventing muscle hypofunction disease or mitochondrial hypofunction disease, and a method for screening a substance involved in a formation of mitochondrial respiratory chain supercomplex.

### Background Art

Mitochondria are organelles present in almost all eukaryotic cells, where aerobic respiration takes place. They are mainly responsible for the production of ATP, which is the energy necessary for life activities, through oxidative phosphorylation. Mitochondrial dysfunction causes muscle dysfunction such as sarcopenia and mitochondrial diseases.

On the other hand, in the medical field in Japan, where the population aging rate is 28.4%, it is an important issue to bring life expectancy and “healthy life expectancy” closer together, i.e., to extend the period during which elderly people can live independently without the need for nursing or medical care. Motor system disorders such as falls, fractures, joint diseases, and weakness account for a large proportion (36.5%) of the causes of nursing care for the elderly. Therefore, regarding the extension of healthy life expectancy, concepts such as “locomotive syndrome” and “frailty” have been proposed. Among these, a prevention and treatment of sarcopenia (age-related decline in skeletal muscle mass and strength) is a core problem. One of the causes of sarcopenia is considered to be the decline in muscle quality associated with the decline in mitochondrial function. The sarcopenia is prevented or treated by dietary and exercise interventions. However, effective intervention methods by drug therapy in advanced stages of sarcopenia have not yet been established. The invention of such a method would be useful in maintaining and improving the health of healthy people and animals.

In addition, mitochondrial diseases are caused by abnormalities of nuclear and mitochondrial genes which encode proteins in mitochondria. In the mitochondrial diseases, various symptoms in nervous tissues and other tissues throughout the body from childhood, with fatigue and muscle weakness in skeletal muscles being the major symptoms. In mitochondrial diseases, various symptoms occur in nervous and other tissues throughout the body from childhood, and an easy fatigability and muscle weakness in skeletal muscles are the major symptoms. At present, taurine is only used for some forms of mitochondrial diseases, and the treatment options for mitochondrial diseases are extremely limited.

### CITATION LIST

### Non-Patent Literature

[NON-PATENT LITERATURE 1] EMBO Journal, 2000 (Germany) Vol. 19, p 1777-1783

### SUMMARY OF INVENTION

### Technical Problem

Therefore, the object of the present invention is to provide a drug for treating or preventing diseases and the like related to a mitochondrial function.

### Solution to Problem

The present inventors have conducted intensive studies for a drug for treating or preventing diseases and the like related to a mitochondrial function, as a result, surprisingly, found that syk inhibitors can promote mitochondrial functions.

The present invention is based on the above findings.

Therefore, the present invention relates to:
[1] an agent for promoting formation of mitochondrial respiratory chain supercomplex, comprising Syk inhibitor as an active ingredient,
[2] the agent for promoting formation of mitochondrial respiratory chain supercomplex of the item [1], wherein the Syk inhibitor is a double-stranded nucleic acid having an RNAi effect on a Syk gene,
[3] the agent for promoting formation of mitochondrial respiratory chain supercomplex of the item [2], wherein the double-stranded nucleic acid is siRNA of an oligonucleotide comprising the base sequences represented by SEQ ID Nos: 1 and 2, or siRNA of an oligonucleotide comprising the base sequences represented by SEQ ID Nos: 3 and 4,
[4] the agent for promoting formation of mitochondrial respiratory chain supercomplex of the item [1], wherein the Syk inhibitor is a compound represented by the following formula (1) or salt thereof:

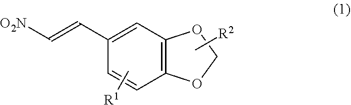

wherein R¹ and R² are independently a hydrogen atom, an alkyl group having 1 to 6 carbon atoms, or an alkoxy group having 1 to 6 carbon atoms, or a compound represented by the following formula (2) or (3), or salt thereof:

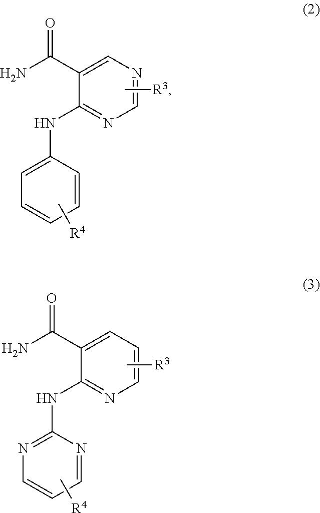

wherein, R³ is any of 1 to 4, which is independently a hydrogen atom, an alkyl group having 1 to 6 carbon atoms, an alkoxy group having 1 to 6 carbon atoms, an optionally substituted —NH-cycloalkyl group, an optionally substituted —NH-heterocycloalkyl group, an optionally substituted —NH-aryl group, or an optionally substituted —NH-alkylenaryl group, or two R³ taken together with two elements to which they are attached may form a 5-membered or 6-membered heterocyclic ring, and
R⁴ is any of 1 to 5, which is independently a hydrogen atom, an alkyl group having 1 to 6 carbon atoms, an alkoxy group having 1 to 6 carbon atoms, an aromatic heterocyclic group, or an optionally substituted aryl group, or two R⁴ taken together with two elements to which they are attached may form a 5-membered or 6-membered heterocyclic ring,
[5] the agent for promoting formation of mitochondrial respiratory chain supercomplex of the item [4], wherein the Syk inhibitor is 3,4-methylenedioxy-β-nitrostyrene represented by the following formula (4):

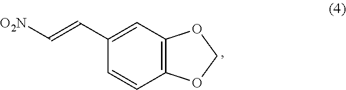 [SMILES: O=[N+]([O-])/C=C/c1ccc2c(c1)OCO2]

2-[[7-(3,4-dimethoxyphenyl)imidazo[1,2-c]pyrimidin-5-yl]amino]pyridine-3-carboxamide represented by the following formula (5):

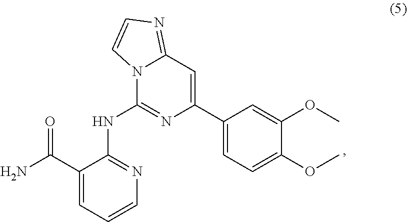 [SMILES: COc1ccc(-c2cc3nccn3c(Nc3ncccc3C(N)=O)n2)cc1OC]

or 2-1[[(3R,4R)-3-aminotetrahydro-2H-pyran-4-yl]amino]-4-[(4-methylphenyl)amino]-5-pyrimidinecarboxamide represented by the following formula (6):

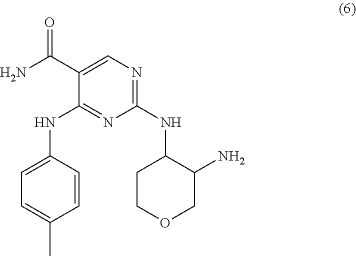 [SMILES: Cc1ccc(Nc2nc(NC3CCOCC3N)ncc2C(N)=O)cc1]

[6] a composition for maintaining or enhancing muscle strength, comprising an agent for promoting formation of mitochondrial respiratory chain supercomplex of any one of items [1] to [5],
[7] A pharmaceutical composition for treating or preventing muscle hypofunction disease or mitochondrial hypofunction disease, comprising an agent for promoting formation of mitochondrial respiratory chain supercomplex of any one of items [1] to [5],
[8] a combination of:
a donor fusion gene in which a gene encoding a FRET donor is fused to a gene encoding at least one protein constituting mitochondrial respiratory chain complex I, a gene encoding at least one protein constituting mitochondrial respiratory chain complex III, a gene encoding at least one protein constituting mitochondrial respiratory chain complex IV, or a COX7RP gene, and
an acceptor fusion gene in which a gene encoding a FRET acceptor is fused to a gene other than the gene to which the gene encoding the FRET donor is fused (with the proviso that the genes to which the FRET donor and FRET acceptor fuse are not genes that encode proteins constituting a single mitochondrial respiratory chain complex),
[9] a combination of a vector comprising the donor fusion gene of the item [8], and a vector comprising the acceptor fusion gene of the item [8],
[10] a cell comprising the combination of the fusion proteins of the item [8], or the combination of the vectors of the item [9],
[11] A method for screening a substance involved in a formation of mitochondrial respiratory chain supercomplex, comprising the steps of:
contacting the cell of the item [10] with a substance to be tested, and measuring a Förster resonance energy transfer.

### Advantageous Effects of Invention

The agent for promoting formation of mitochondrial respiratory chain supercomplex of the present invention can be used to maintain or enhance muscle strength, or to treat muscle hypofunction disease or mitochondrial hypofunction disease. In addition, the screening method of the present invention can discover substances associated with improvement or deterioration of mitochondrial function.

### BRIEF DESCRIPTION OF THE DRAWINGS

FIG. 1 is a view (A) schematically showing mitochondrial respiratory chain complexes I, II, III, and IV, and supercomplex I+III2+IVn(n:1-4), a view (B) schematically showing a composition of supercomplex I+III2+IVn(n:1-4), and a view (C) schematically showing a generation of FRET between a fusion protein of complex I subunit (NDUFB8) and AcGFP, and a fusion protein of complex IV subunit (COX8A) and DsRed Monomer.

FIG. 2 is a fluorescence micrograph (A) of C2C12 cells in which plasmid vectors of NDUFB8 (complex I)-AcGFP, UQCR11 (complex III)-AcGFP, COX8A (complex IV)-DsRed monomer, and ATP5F1c-DsRed monomer were introduced thereinto, a fluorescence micrograph (B) showing a generation of FRET in C2C12 cells in which NDUFB8-AcGFP and COX8A-DsRed monomer were co-expressed, and a graph (C) showing an Acceptor Photobleaching.

FIG. 3 is photographs of a generation of FRET in fixed cells (A), or live cells (B) of C2C12 cells in which NDUFB8-AcGFP and COX8A-DsRed monomer were stably expressed.

FIG. 4 is photographs (A) showing a decrease in FRET efficiency in C2C12 cells in which the expression of COX7RP, which is known to promote the formation of respiratory chain supercomplexes, was suppressed with siRNA, a graph (B) showing Corrected FRET(cFRET)/Donor, and photographs (C) of electrophoresis showing decreased formation of respiratory chain supercomplexes I+III2+IVn(n:1-2), 1+1112, and 1112+IV.

FIG. 5 is a view (A) showing the screening process for substances that affect the formation of respiratory chain supercomplexes using C2C12 cells in which NDUFB8-AcGFP and COX8A-DsRed monomer were stably expressed, a graph (B) showing corrected cFRET/Donor value for screened 1280 substances, a dose-response curve (C) of the resulting MNS, a photograph (D) showing the increase in FRET efficiency between subunit fluorescent fusion proteins of respiratory chain complexes I and IV by the addition of MNS, a graph (E) showing improvement in corrected cFRET/Donor value by the addition of MNS, photographs (F) showing increased formation of respiratory chain supercomplexes I+III2+IV, I+III2, III2+IV by the addition of MNS, and a graph (G) showing oxygen consumption upon the addition of MNS.

FIG. 6 is graphs (A, B) showing dose-response curves of Syk inhibitors, i.e. BAY61-3606 and GSK143, respectively, photographs (C) showing increased formation of respiratory chain supercomplexes I+III2+IV, I+III2, III2+IV by the addition of BAY61-3606 and GSK143, and graphs (D, E) showing oxygen consumption when adding BAY61-3606 and GSK143, respectively.

FIG. 7 is a graph showing FRET efficiency (A), photographs (B) showing the formation of respiratory chain supercomplex I+III2+IVn(n:1-2), I+III2, III2+IV, and graphs (C, D) showing oxygen consumption, when C2C12 cells were treated with siRNA against Syk.

FIG. 8 is views showing changes in body weight (toxicity) (a), wire hang test (b), treadmill test (c-e), formations of respiratory chain supercomplex I+III2+IVn(n=1-2), I+III2, III2+IV (f) and expression of COX7RP (g) in the extensor digitorum longus and soleus muscles, when MNS was administered to mice.

FIG. 9 is views showing changes in body weight (toxicity) (a), wire hang test (b), and treadmill test (c-e) when BAY61-3606 or GSK143 was administered to mice.

### ADVANTAGEOUS EFFECTS OF INVENTION

### [1] Screening Method

The method for screening a substance involved in a formation of mitochondrial respiratory chain supercomplex comprises the steps of contacting the cell in which the supercomplex formation can be determined by FRET with a substance to be tested, and measuring a Förster resonance energy transfer.

### <<Cells for Determining Mitochondrial Respiratory Chain Supercomplex Formation by FRET>>

Cells for determining mitochondrial respiratory chain supercomplex formation by FRET comprises (a) combination of: a donor fusion gene in which a gene encoding a FRET donor is fused to a gene encoding at least one protein constituting mitochondrial respiratory chain complex I, a gene encoding at least one protein constituting mitochondrial respiratory chain complex III, a gene encoding at least one protein constituting mitochondrial respiratory chain complex IV, or a COX7RP gene, and an acceptor fusion gene in which a gene encoding a FRET acceptor is fused to a gene other than the gene to which the gene encoding the FRET donor is fused (with the proviso that the genes to which the FRET donor and FRET acceptor fuse are not genes that encode proteins constituting a single mitochondrial respiratory chain complex), or (b) combination of a vector comprising the donor fusion gene, and a vector comprising the acceptor fusion gene.

### <<Mitochondrial Respiratory Chain Supercomplex>>

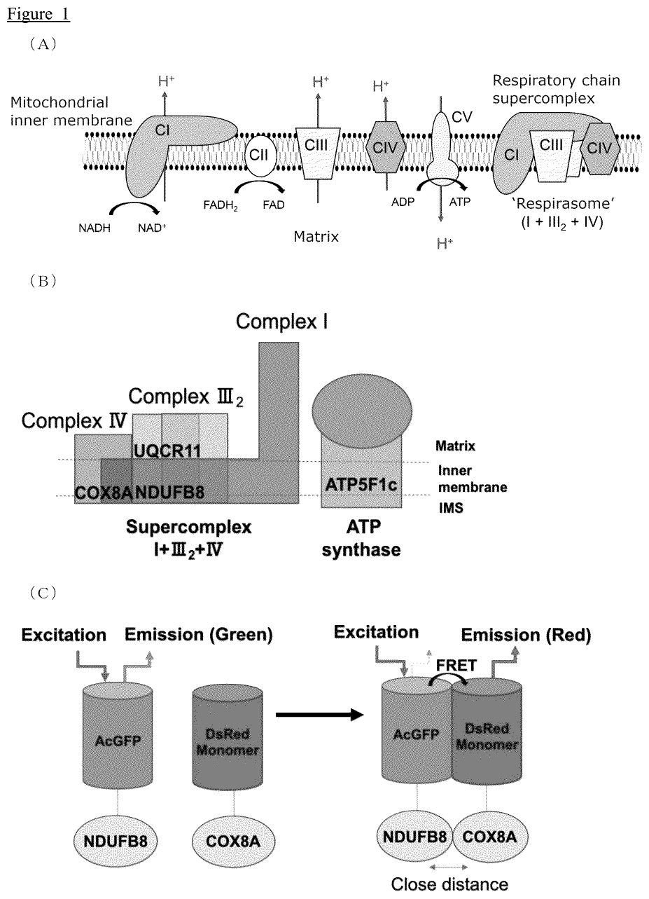

The mitochondrial respiratory chain complex is composed of four different complexes, i.e. complexes I, II, III, and IV. Furthermore, the above complexes forms three type of supercomplexes, i.e. I+III2 consisting of complex I, and dimeric complex III; I+III2+IVn(n:1-4) consisting of complex I, dimeric complex III, and monomeric to tetrameric complex IV; III2+IVn(n:1-2) consisting of dimeric complex III and monomeric or dimeric complex IV. In particular, the supercomplex I+III2+IVn (n:1-4), which comprises all complexes I, III and IV, is called respirasome (FIG. 1A).

The mitochondrial respiratory chain complex I is composed of subunits (proteins) consisting of NDUFS7, NDUFS8, NDUFV2, NDUFS3, NDUFS2, NDUFV1, NDUFS1, ND1, ND2, ND3, ND4, ND4L, ND5, ND6, NDUFS6, NDFUA12, NDUFS4, NDUFA9, NDUFAB1, NDUFA2, NDUFA1, NDUFB3, NDUFA5, NDUFA6, NDUFA11, NDUFB11, NDUFS5, NDUFB4, NDUFA13, NDUFB8, NDUFA8, NDUFB9, NDUFA8, NDUFB9, NDUFB10, NDUFB8, NDUFC2, NDUFB2, NDUFA7, NDUFA3, NDUFA4, NDUFB5, NDUFB1, NDUFC1, NDUFA10, NDUFA4L2, NDUFV3, and NDUFB6. The mitochondrial respiratory chain complex III is composed of subunits (proteins) consisting of MT-CYB, CYC1, Rieske, UQCR1, UQCR2, UQCR6, UQCR7, UQCR8, UQCR9, and UQCR10. The mitochondrial respiratory chain complex IV is composed of subunits (proteins) consisting of COX1, COX2, COX3, COX4I1, COX4A2, COX5A, COX5B, COX6A1, COX6A2, COX6B1, COX6B2, COX6C, COX7A1, COX7A2, COX7B, COX7C, COX8A, and COX8C. Further, a respiratory chain complex-promoting factor, COX7RP, is not a subunit that forms complexes I, II, III, and IV, but it binds to complexes I, III, and IV and promotes the formation of supercomplex.

### (Donor Fusion Gene and Acceptor Fusion Gene)

In the donor fusion gene, a gene encoding a FRET donor is fused to a gene encoding at least one protein constituting mitochondrial respiratory chain complex I, a gene encoding at least one protein constituting mitochondrial respiratory chain complex III, a gene encoding at least one protein constituting mitochondrial respiratory chain complex IV, or a COX7RP gene.

Further, in the acceptor fusion gene, a gene encoding a FRET acceptor is fused to a gene encoding at least one protein constituting mitochondrial respiratory chain complex I, a gene encoding at least one protein constituting mitochondrial respiratory chain complex III, a gene encoding at least one protein constituting mitochondrial respiratory chain complex IV, or a COX7RP gene.

The gene in which the gene encoding FRET donor is fused and the gene in which the gene encoding FRET acceptor is fused, are different. Further, the gene in which the gene encoding FRET donor is fused and the gene in which the gene encoding FRET acceptor is fused, are genes encoding proteins (subunits) constituting different mitochondrial respiratory chain complexes.

In particular, when the gene in which the gene encoding FRET donor is fused, is a gene that encode protein constituting complex I, the gene in which the gene encoding FRET acceptor is fused, is a gene that encode protein constituting complex III or complex IV, or a gene that encode COX7RP. When the gene in which the gene encoding FRET donor is fused, is a gene that encode protein constituting complex IV, the gene in which the gene encoding FRET acceptor is fused, is a gene that encode protein constituting complex I or complex III, or a gene that encode COX7RP. When the gene in which the gene encoding FRET donor is fused, is a gene that encode COX7RP, the gene in which the gene encoding FRET acceptor is fused, is a gene that encode protein constituting complex I, complex III, or complex IV.

That is to say, the FRET donor fusion protein and the RET acceptor fusion protein which are fused with proteins constituting different complexes respectively, are expressed in cells, and whereby the formation of mitochondrial respiratory chain supercomplexes can be measured by Forster resonance energy transfer.

The formation of I+III2 supercomplex, I+III2+IVn(n:1-4) supercomplex, or III2+IVn(n:1-2) supercomplex can be measured by the combination of the FRET donor fusion gene and the FRET acceptor fusion genes.

When the I+III2 supercomplex is measured, preferably the gene encoding the FRET donor is fused to the gene encoding the protein constituting complex I or complex III, and the gene encoding the FRET acceptor is fused to the gene encoding the protein constituting the other complex. When the III2+IVn(n:1-2) supercomplex is measured, preferably the gene encoding the FRET donor is fused to the gene encoding the protein constituting complex III or complex IV, and the gene encoding the FRET acceptor is fused to the gene encoding the protein constituting the other complex. When the I+III2+IVn(n:1-4) supercomplex is measured, preferably the gene encoding the FRET donor is fused to the gene encoding the protein constituting complex I, complex III or complex IV, and the gene encoding the FRET acceptor is fused to the gene encoding the protein constituting the other complex. Furthermore, the supercomplex can be detected when one of the genes encoding the FRET donor or the FRET acceptor is fused to the gene encoding the COX7XR protein and the other to the genes encoding the protein constituting either complex.

### (Forster Resonance Energy Transfer)

Forster resonance energy transfer is an interaction between two or more molecules with different electronic excitation states, in which one molecule (donor molecule) is excited by an external light source and then transfers its energy to the other molecule (acceptor molecule). Forster resonance energy transfer includes fluorescence resonance energy transfer using fluorescent molecules as donor and acceptor molecules, and bioluminescence resonance energy transfer (hereinafter sometimes referred to as BRET) using bioluminescent and fluorescent molecules as donor and acceptor molecules, respectively.

The proteins to be translated from the donor and acceptor genes are donor and acceptor molecules. The donor and acceptor molecules are different from each other. In this case, FRET is detected, for example, by the appearance of increased fluorescence of the acceptor or by quenching of fluorescence from the donor molecule. In order to achieve FRET, the donor molecule must be able to absorb light and transfer energy to the acceptor molecule through resonance of the excited electrons. Furthermore, in order to generate FRET, the fluorescence emission wavelength of the donor molecule should be the excitation wavelength of the acceptor molecule. Fluorescent compounds, fluorescent proteins, or bioluminescent proteins can be used as donor molecules, and fluorescent compounds or fluorescent proteins can be used as acceptor molecules. In other words, as a combination of donor and acceptor molecules, a fluorescent compound and a fluorescent compound, a fluorescent compound and a fluorescent protein, a fluorescent protein and a fluorescent compound, a fluorescent protein and a fluorescent protein, a bioluminescent protein and a fluorescent compound, or a bioluminescent protein and a fluorescent protein can be used.

### (Fluorescent Compound)

Fluorescent compounds include fluoresceins such as carboxyfluorescein, 6-(fluorescein)-5,6-carboxamidohexanoic acid or fluorescein isothiocyanate; Alexa Fluor dyes such as Alexa Fluor 488 or Alexa Fluor 594; cyanine dyes such as Cy2, Cy3, Cy5 or Cy7, coumarin; R-phycoerythrin; allophycoerythrin; and modified allophycocyanines such as XL665; texas red, princeton red, phycobiliprotein, europium cryptate, x1665, avidin streptavidin, rhodamine, eosin, erythrosine, naphthalene, pyrene, pyridyloxazole, benzoxadiazole and sulfoindocyanine, and derivatives thereof or complexes thereof. In the present invention, a combination that satisfies the above requirements and enables FRET, can be selected for use, among the above donor and acceptor molecules.

Examples of suitable combinations of the above fluorescent compounds used in the present invention may include Rhodamine B sulfonyl chloride and fluorescein maleimide; N-iodoacetyl-N′-(5-sulfo-1-naphthyl)ethyl-endiamine (1,5-IAEDANS) or Iodoacetamide, and succinimidyl 6-(N-(7-nitrobenzo-2-oxa-1,3-diazol-4-yl)amino)hexanoate (NBD-X,SE); (diethylamino)coumarin (DEAC) or N-methyl-anthraniloyldeoxyguanine nucleotide (e.g. MantdGDP or MantdGTP) and sNBD (succinimidyl 6-[(7-nitrobenzo-2-oxa-1,3-diazol-4-yl)amino]hexanoate), or the like.

### (Vector)

A vector used in the combination of the vector comprising the donor fusion gene and the vector comprising the acceptor fusion gene of the present invention is not limited. Any vectors such as plasmids, phages, or viruses can be used. Examples of a plasmid for Escherichia coli may include pBR322, pBR325, pUC118, pUC119, pKC30, or pCFM536, a plasmid for Bacillus subtilis, such as pUB110, a plasmid for yeast, such as pG-1, YEp13, or YCp50, phage DNA such as λgt110, or λZAPII. Further, examples of the vector for mammalian cells may include a virus DNA such as baculovirus, vaccinia virus, or adenovirus; SV40; or derivatives thereof. The vector comprises an initiation site of replication, a selective marker, and a promotor, and if necessary may comprise an enhancer, a terminator, a ribosomal binding site, and polyadenylation signal.

### (Cell)

The cells of the present invention are not limited, as long as they can express the donor fusion gene and the acceptor fusion gene contained within the cells, but are cells that express proteins that form mitochondrial complexes other than donor-bound and acceptor-bound proteins. Fungal cells, yeast cells, insect cells, or mammalian cells can be used, but mammalian cells are preferable from the viewpoint of screening substances involved in the formation of supercomplexes.

Mammalian cells include, for example, human-derived cell lines (such as RD cells) or mouse-derived cell lines (such as C2C12 cells). The cells of the present invention can be obtained by introducing the above vectors or the above fusion genes into these cells.

### <<Contact Step(1)>>

In the contact step (1) of the present invention, the cell of the present invention is contacted with a substance to be tested.

The test substance is not particularly limited, but various known compounds (including peptides) registered in chemical files, compounds obtained by the combinatorial chemistry techniques [Terrett, N. K. et al., Tetrahedron, 51, 8135-8137 (1995)], or a group of random peptides prepared by employing a phage display method [Felici, F. et al., J. Mol. Biol., 222, 301-310 (1991)], or low-molecular compounds, can be used. In addition, culture supernatants of microorganisms, culture supernatants of cells, body fluids in the living body, natural components derived from plants or marine organisms, or animal tissue extracts and the like, may be also used as the test substance for screening.

A timing of contact of cells with test substances in the contact step, i.e., a timing of addition of test substances, is not particularly limited.

In addition, a concentration of a test substance may be optionally decided, but it is considered that there may be an optimal concentration of the test substance. Thus, it is preferable that the test substance diluted in a stepwise fashion is used. A medium used in the contact step can be appropriately selected according to the cells used.

### <<FRET Measuring Step (2)>>

### (2) Step for Measuring a Forster Resonance Energy Transfer

In the FRET measuring step, the occurrence of Förster resonance energy transfer can be detected by an enhancement of fluorescence emission from the acceptor or by a fluorescence quenching from the donor molecule. By detecting and/or measuring the occurrence of FRET, the formation of mitochondrial respiratory chain supercomplexes can be detected and/or measured.

### [2] Agent for Promoting Formation of Mitochondrial Respiratory Chain Supercomplex

The agent for promoting formation of mitochondrial respiratory chain supercomplex of the present invention comprises, Syk (spleen associated tyrosine kinase) inhibitor as an active ingredient.

Syk inhibitors are not limited as long as they suppress and/or inhibit the activity of Syk, but include, for example, double-stranded nucleic acids with RNAi effects, anti-Syk antibodies, Syk inhibitory compounds, or the like. Inhibition or suppression of Syk activity includes, but is not limited to, suppression of Syk mRNA expression, suppression of Syk protein expression, and suppression or inhibition of Syk protein function, or the like.

<<Double-Stranded Nucleic Acid with RNAi Effect>>

The double-stranded nucleic acid is a double-stranded nucleic acid having an RNAi effect on the Syk gene, and is characterized in that it comprises a sense strand comprising a nucleotide sequence corresponding to, for example, the human SyK target sequence of SEQ ID No: 17 or the mouse SyK target sequence of SEQ ID No: 18, and an antisense strand comprising a complementary nucleotide sequence to the above sense strand.

In this specification, the term “double-stranded nucleic acid” means a nucleic acid molecule comprising a double-stranded nucleic acid region in which the desired sense strand and antisense strand are hybridized, and preferably it is siRNA (small interfering RNA).

The double-stranded nucleic acid of the present invention comprises a sense strand comprising a nucleotide sequence corresponding to the target sequence of SEQ ID No: 17 or 18 and an antisense strand comprising a nucleotide sequence complementary to the above sense strand. In this specification, the term “nucleotide sequence corresponding to target sequence” means a nucleotide sequence identical to the target sequence, or a nucleotide sequence in which one or several bases (for example, 2 to 3 bases) are substituted in the target sequence. In the case that the double-stranded nucleic acid is siRNA, it is known that RNAi effect can be obtained even if siRNA comprises mismatches of one to several bases. In the present invention, not only sequences identical to the target sequence but also sequences comprising mismatches may be acceptable, as long as the RNAi effect is obtained.

In addition, the “complementary sequence to the sense strand” in the antisense strand may be a sequence that is complementary to the sense strand such that it may hybridize with the sense strand. That is to say, it is a base sequence that can be completely complementary to the sense strand or a base sequence in which one or several bases (for example, 2-3 bases) are replaced in the sequence that is completely complementary to the sense strand.

### (Types and Modifications of Nucleic Acid)

The types of nucleic acids constituting double-stranded nucleic acids are not particularly limited and can be appropriately selected. Examples of the types of nucleic acids may include a double-stranded RNA or a DNA-RNA chimeric double-stranded nucleic acid. In the chimeric double-stranded nucleic acids, a part of double-stranded RNA with RNAi effect is replaced with DNA, and it is known that it has high stability in serum and low inducibility of immune responses.

In addition, in the double-stranded nucleic acids, a resistant and stable against nucleases may be increased, for example, by modifying the 2′—OH group, by substituting using phosphorothioates, or modifying using boranophosphate groups in the backbone, or by introducing LNA (locked nucleic acid) in which the 2 and 4 positions of ribose are cross-linked, or the like. Alternatively, for the purpose of increasing the efficiency of introduction into cells, the 5′ or 3′ end of the sense strand of the double-stranded nucleic acid can be modified with, for example, nanoparticles, cholesterol, or peptides that pass through cell membrane.

(siRNA)

The double-stranded RNA of the present invention is preferably siRNA (including chimeric RNA). The “siRNA” is a small molecular double-stranded RNA of 18 to 29 bases (preferably 21 to 23 bases) in length, which has the function of suppressing the expression of the target gene by cleaving the mRNA of the target gene that has a complementary sequence to the antisense strand (guide strand) of the siRNA. In other words, siRNA can destroy messenger RNA (mRNA) by RNA interference (RNAi) and suppress gene expression in a sequence-specific manner. A base sequence of siRNA can be appropriately designed based on the base sequence of Syk mRNA (base sequence corresponding to the RNA of SEQ ID No: 17 or 18). A terminal structure of the siRNA is not particularly limited, as long as the siRNA comprises sense strand and antisense strand as described above and exhibits the desired RNAi effect, and thus it can be selected as appropriate. For example, the siRNA may have a blunt end or a protruding end (overhang). Among them, the siRNA preferably has a structure in which the 3′ end of each strand protrudes by 2-6 bases, and more preferably has a structure in which the 3′ end of each strand protrudes by 2 bases. In addition, siRNA made from the mRNA base sequence of Syk can be used as an active ingredient in the agent for promoting formation of mitochondrial respiratory chain supercomplex, or pharmaceutical composition for treating or preventing muscle hypofunction disease or mitochondrial hypofunction disease, regardless of the degree of effectiveness.

Examples of siRNA of the present invention, as shown in Table 1, may include siRNA comprising a sense strand of SEQ ID NO:1 (21 bases) and an antisense strand of SEQ ID NO:2 (21 bases) (siSyk #1 in the example described below), and siRNA comprising a sense strand of SEQ ID NO:3 (21 bases) and an antisense strand of SEQ ID NO:4 (21 bases) (siSyk #2 in the example described below), which target the sequence of SEQ ID NO: 5 (23 bases).

TABLE 1

| | Target | |
|---|---|---|
| siRNA | sequence | Base sequence |
| #1 | 4090-4112 | 5′-AAUGAAUUCAACAUACAGGGA-3′ (SEQ ID NO: 1) |
| | | 5′-CCUGUAUGUUGAAUUCAUUGA-3′ (SEQ ID NO: 2) |
| #2 | 4519-4541 | 5′-UUAAUCUUGACAGUAAGACAC-3′ (SEQ ID NO: 3) |
| | | 5′-GUCUUACUGUCAAGAUUAAUU-3′ (SEQ ID NO: 4) |

(Target sequence)
#1: UCAAUGAAUUCAACAUACAGGGA(4090-4112) (SEQ ID NO 5)
#2: AAUUAAUCUUGACAGUAAGACAC(4519-4541) (SEQ ID NO 6)

### (Preparing Method)

The double-stranded RNA (especially siRNA) of the present invention can be prepared based on conventionally known methods.

For example, single-stranded RNAs of 18 to 29 bases in length corresponding to each of the desired sense and antisense strands, can be chemically synthesized using existing automated DNA/RNA synthesizers and then annealed.

In addition, siRNA can be prepared using an intracellular reaction by constructing a desired siRNA expression vector such as the vector of the present invention described below, and introducing the expression vector into the cell.

It is preferable that the DNA comprised in the vector is linked to a promoter sequence for controlling the transcription of the double-stranded nucleic acid, at upstream (5′ side) of the base sequence encoding said double-stranded nucleic acid. The promoter sequence is not particularly limited, and can be appropriately selected. Examples of the promoter may include pol II promoters such as CMV promoter, pol III promoters such as H1 promoter and U6 promoter, and the like. Furthermore, it is preferable that a terminator sequence for terminating the transcription of the double-stranded nucleic acid is linked thereto at downstream (3′ side) of the nucleotide sequence encoding the double-stranded nucleic acid. The terminator sequence is not particularly limited, and can be appropriately selected according to the purpose.

The vector is not particularly limited, as long as it comprises the DNA, and can be appropriately selected according to the purpose. Examples of the vector may include plasmid vectors and viral vectors. It is preferable that the vector is an expression vector capable of expressing the double-stranded nucleic acids (especially siRNA).

The method of expression of the double-stranded nucleic acid is not particularly limited, and can be appropriately selected according to the purpose.

Examples of the method of expressing siRNA as double-stranded nucleic acid may include an expression method for expressing two short single-stranded RNAs (tandem type), and an expression method for single-stranded RNA as shRNA (short hairpin RNA) (hairpin type), and so on. The shRNA is a single-stranded RNA that comprises a dsRNA region of about 18 to 29 bases and a loop region of about 3 to 9 bases. The shRNA forms base pairs to be hairpin-shaped double-stranded RNA when it is expressed in vivo. Then, the shRNA is cleaved by Dicer (RNase III enzyme) to form siRNA, which can function to suppress the expression of target genes.

The tandem-type siRNA expression vector comprises a DNA sequence encoding a sense strand and a DNA sequence encoding an antisense strand of siRNA, and a promoter sequence is linked to the upstream (5′ side) of the DNA sequence encoding each strand. Further, a terminator sequence is linked to the downstream (3′ side) of the DNA sequence encoding each strand.

In the hairpin-type siRNA expression vector, a DNA sequence encoding the sense strand and a DNA sequence encoding the antisense strand of siRNA are arranged in the opposite direction. The sense strand DNA sequence and the antisense strand DNA sequence are connected via a loop sequence, and a promoter sequence is connected upstream (5′ side) and a terminator sequence is connected downstream (3′ side) thereof.

### <<Syk Inhibitory Compound>>

The Syk inhibitor is not particularly limited as long as it can suppress an expression of Syk or an function of Syk. However, examples of the Syk inhibitor may include a compound represented by the following formula (1) or salt thereof:

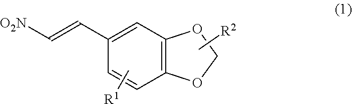

wherein R¹ and R² are independently a hydrogen atom, an alkyl group having 1 to 6 carbon atoms, or an alkoxy group having 1 to 6 carbon atoms (hereinafter sometimes referred to as compound A), or
a compound represented by the following formula (2) or (3), or salt thereof:

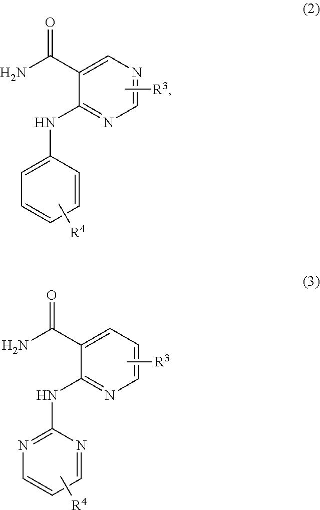

wherein, R³ is any of 1 to 4, which is independently a hydrogen atom, an alkyl group having 1 to 6 carbon atoms, an alkoxy group having 1 to 6 carbon atoms, an optionally substituted —NH-cycloalkyl group, an optionally substituted —NH-heterocycloalkyl group, an optionally substituted —NH-aryl group, or an optionally substituted —NH-alkylenaryl group, or two R³ taken together with two elements to which they are attached may form a 5-membered or 6-membered heterocyclic ring, and
R⁴ is any of 1 to 5, which is independently a hydrogen atom, an alkyl group having 1 to 6 carbon atoms, an alkoxy group having 1 to 6 carbon atoms, an aromatic heterocyclic group, or an optionally substituted aryl group, or two R⁴ taken together with two elements to which they are attached may form a 5-membered or 6-membered heterocyclic ring (hereinafter sometimes referred to as compound B).

Specifically, the examples of an alkyl group having 1 to 6 carbon atoms include a methyl group, an ethyl group, a normal propyl group, an isopropyl group, a normal butyl group, an isobutyl group, a secondary butyl group, a tertiary butyl group, a normal pentyl group, an isopentyl group, a neopentyl group, a tertiary pentyl group, a normal hexyl group, and an isohexyl group. An alkyl group having 1 to 3 carbon atoms is preferred.

Specifically, the examples of an alkoxy group having 1 to 6 carbon atoms include a methoxy group, an ethoxy group, a normal propoxy group, an isopropoxy group, a normal butoxy group, an isobutoxy group, a secondary butoxy group, a tertiary butoxy group, a normal pentyloxy group, an isopentyloxy group, a neopentyloxy group, a normal hexyloxy group, or an isohexyloxy group. An alkoxy group having 1 to 3 carbon atoms is preferred.

The optionally substituted —NH-cycloalkyl group means a group in which one substituent of the secondary amine is a cycloalkyl group. The cycloalkyl group is preferably a cycloalkyl group having 3 to 8 carbon atoms, and more preferably a cycloalkyl group having 5 to 7 carbon atoms. Specifically, examples of the cycloalkyl group may include a cyclopropyl group, a cyclobutyl group, a cyclopentyl group, a cyclohexyl group, a cycloheptyl group, or a cyclooctyl group. Substituents include C₁₋₆ alkyl group, C₁₋₆ alkoxy group, amino group, hydroxyl group, carboxy group, C₃₋₈ cycloalkyl group, C₂₋₆ alkynyl group, saturated or unsaturated C₁₋₆ hydrocarbon group with oxygen atom and carbonyl group.

The optionally substituted —NH-heterocycloalkyl group means a group in which one substituent of the secondary amine is a heterocycloalkyl group. The heterocycloalkyl group is preferably a heterocycloalkyl group having 3 to 8 carbon atoms, more preferably a heterocycloalkyl group having 5 to 7 carbon atoms. Examples of a hetero atom may include oxygen atom, nitrogen atom, or sulfur atom, but oxygen atom is preferable.

Specifically, examples of the heterocycloalkyl group may include pyrrolidine, piperidine, pyrazolidine, imidazolidine, piperazine (1,2-, 1,3- and 1,4-isomer), oxazolidine, isoxazolidine, thiazolidine, isothiazolidine, morpholine or thiomorpholine. Examples of the heterocycloalkyl group containing an oxygen atom may include a cyclopropyl group, a cyclobutyl group, a cyclopentyl group, a cyclohexyl group, a cycloheptyl group, or a cyclooctyl group, in which one or two carbon atoms are substituted with an oxygen atom. Substituents include C₁₋₆ alkyl group, C₁₋₆ alkoxy group, amino group, hydroxyl group, carboxy group, C₃₋₈ cycloalkyl group, C₂₋₆ alkynyl group, saturated or unsaturated C₁₋₆ hydrocarbon group with oxygen atom and carbonyl group.

The optionally substituted —NH-aryl group means a group in which one substituent of the secondary amine is an aryl group. The aryl group is preferably an aryl group having 6 to 10 carbon atoms. Specifically, examples of the aryl group may include a phenyl group or a naphthyl group, and a phenyl group is preferable. Substituents include C₁₋₆ alkyl group, C₁₋₆ alkoxy group, amino group, hydroxyl group, carboxy group, C₃₋₈ cycloalkyl group, C₂₋₆ alkynyl group, saturated or unsaturated C₁₋₆ hydrocarbon group with oxygen atom and carbonyl group.
a. The optionally substituted —NH— alkylenaryl group means a group in which one substituent of the secondary amine is an alkylenaryl group.

The alkylene aryl group is preferably an alkylene group in which an alkylene group having 1 to 6 carbon atoms, bonded to one carbon atom of the aryl group, and more preferably an alkylene group in which an alkylene group having 1 to 3 carbon atoms, bonded to one carbon atom of the aryl group. The aryl group includes the aryl group in the —NH-aryl group described above. Substituents include C₁₋₆ alkyl group, C₁₋₆ alkoxy group, amino group, hydroxyl group, carboxy group, C₃₋₈ cycloalkyl group, C₂₋₆ alkynyl group, saturated or unsaturated C₁₋₆ hydrocarbon group with oxygen atom and carbonyl group.

The aromatic heterocyclic group means a group in which one hydrogen atom is removed from a 5 to 8 membered aromatic heterocyclic group or a fused ring group thereof, i.e. an aromatic heterocyclic group containing a heteroatom in the ring or a fused ring group thereof. Heteroatoms include an oxygen atom, a sulfur atom, a nitrogen atom, a phosphorus atom, a boron atom, or an arsenic atom. Specifically, examples of the aromatic heterocyclic groups or fused rings may include a triazole group, a pyridyl group, a pyrazyl group, a pyrimidyl group, a quinolyl group, an isoquinolyl group, a pyrrolyl group, an indolenyl group, an imidazolyl group, a carbazolyl group, a thienyl group, or a furyl group

The optionally substituted aryl group is preferably an aryl group having 6 to 10 carbon atoms. Specifically, examples of the optionally substituted aryl group may include a phenyl group or a naphthyl group, and a phenyl group is preferable. Substituents include C₁₋₆ alkyl group, C₁₋₆ alkoxy group, amino group, hydroxyl group, carboxy group, C₃₋₈ cycloalkyl group, C₂₋₆ alkynyl group, saturated or unsaturated C₁₋₆ hydrocarbon group with oxygen atom and carbonyl group.

Examples of a five or six-membered heterocyclic ring wherein two R³ and two elements attached thereto together form the ring may include triazole, pyridyl, pyrazyl, pyrimidyl, quinolyl, isoquinolyl, pyrrolyl, indolenyl, imidazolyl, carbazolyl, thieni group, or furyl.

The compound A is a derivative of nitrostyrene. The derivatives of nitrostyrene can be synthesized by various methods. In addition, the Compound B is a derivative of pyridine or pyrimidine. Pyridine or pyrimidine and their derivatives can also be synthesized by various methods. These compounds can also be purchased commercially.

The compound A is not limited, but includes, a compound (3,4-methylenedioxy-p-nitrostyrene; MNS) represented by the following formula (4):

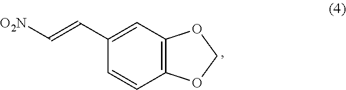 [SMILES: O=[N+]([O-])/C=C/c1ccc2c(c1)OCO2]

In addition, the compound B is not limited, but includes, a compound (2-[[7-(3,4-dimethoxyphenyl)imidazo[1,2-c]pyrimidin-5-yl]amino]pyridine-3-carboxamide) (BAY61-3606) represented by the following formula (5):

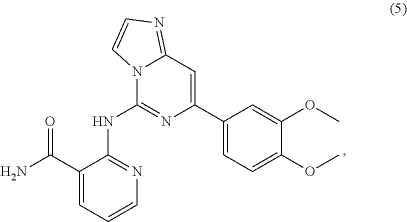 [SMILES: COc1ccc(-c2cc3nccn3c(Nc3ncccc3C(N)=O)n2)cc1OC]

a compound (2-[[(3R,4R)-3-aminotetrahydro-2H-pyran-4-yl]amino]-4-[(4-methylphenyl)amino]-5-pyrimidine carboxamide) (GSK143) represented by the following formula (5):

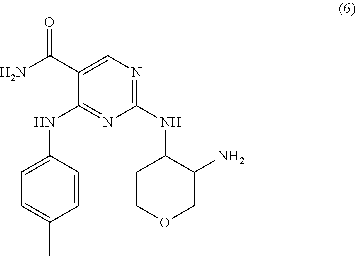 [SMILES: Cc1ccc(Nc2nc(NC3CCOCC3N)ncc2C(N)=O)cc1]

and the compounds represented by the following formulas (7) to (11):

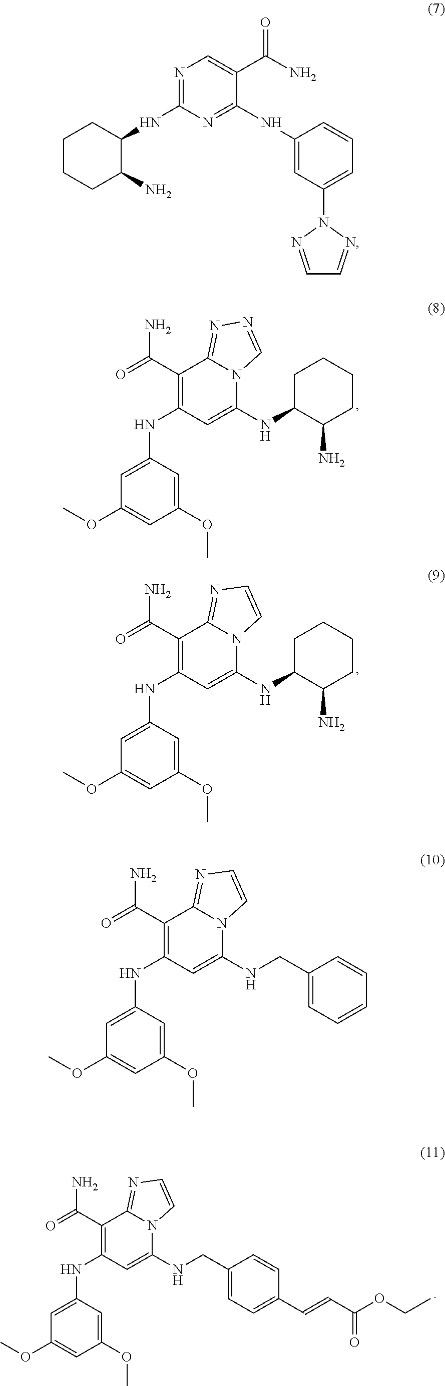

The salts of compound A and compound B are pharmaceutically acceptable salts, which may be acid addition salts or salts with bases, depending on the type of substituent. Specifically, the examples of acid addition salts or salts with bases include acid addition salts with inorganic acids such as hydrochloric acid, hydrobromic acid, hydroiodic acid, sulfuric acid, nitric acid, and phosphoric acid, and organic acids such as formic acid, acetic acid, propionic acid, oxalic acid, malonic acid, succinic acid, fumaric acid, maleic acid, lactic acid, malic acid, mandelic acid, tartaric acid, dibenzoyl tartrate, ditolyl tartrate, citric acid, methanesulfonic acid, ethanesulfonic acid, benzenesulfonic acid, p-toluenesulfonic acid, aspartic acid, and glutamic acid, salts with inorganic bases such as sodium, potassium, magnesium, calcium, and aluminum, and organic bases such as methylamine, ethylamine, ethanolamine, lysine, and ornithine, salts with various amino acids and amino acid derivatives such as acetylleucine, and ammonium salts, etc.

Furthermore, the active ingredients used in the present invention also include various hydrates, solvates, and crystalline polymorphs of compound A or compound B and salts thereof. The present invention also includes compounds labeled with various radioactive or non-radioactive isotopes.

### <<Composition for Maintaining or Enhancing Muscle Strength>>

The composition for maintaining or enhancing muscle strength of the present invention comprises the agent for promoting formation of mitochondrial respiratory chain supercomplex as an active ingredient. It can maintain or enhance muscle strength by promoting formation of mitochondrial respiratory chain supercomplex.

### <<Pharmaceutical Composition>>

The pharmaceutical composition for treating or preventing muscle hypofunction disease comprises an agent for promoting formation of mitochondrial respiratory chain supercomplex as an active ingredient. It can treat or prevent muscle hypofunction disease by promoting formation of mitochondrial respiratory chain supercomplex. Examples of the muscle hypofunction disease may include a sarcopenia, a frailty, a mitochondrial disease, an age-related disease, or the like.

A pharmaceutical composition containing one or more of the above-mentioned compound A or compound B, or salts thereof as an active ingredient can be prepared by using excipients commonly used in the art, that is to say, excipients for pharmaceutical compositions and drug carriers, etc., by a method commonly used in the art.

Administration may be oral in pill, capsule, granule, powder, or liquid form, etc., or parenteral in injection such as intra-articular, intravenous, or intramuscular, suppository, eye drop, eye ointment, transdermal solution, ointment, transdermal patch, transmucosal solution, transmucosal patch, inhalation, etc.

The examples of solid composition for oral administration include tablets, dispersions, and granules. In such solid compositions, one or more active ingredients are mixed with at least one inert excipient, such as lactose, mannitol, glucose, hydroxypropyl cellulose, microcrystalline cellulose, starch, polyvinylpyrrolidone, and/or magnesium metasilicate. The composition may contain inert additives, such as lubricants e.g., magnesium stearate, disintegrants e.g., sodium carboxymethylstatinate, stabilizer, solubilizer, etc., according to conventional methods. Tablets or pills may be coated with a sugar coating or a film of a gastric or enteric soluble substance as needed.

The examples of liquid composition for oral administration include pharmaceutically acceptable emulsion, solution, suspension, syrup, or elixir, which contain commonly used inert diluent, such as purified water or ethanol. In addition to the inert diluent, the liquid composition may contain auxiliary agents such as solubilizer, wetting agent, and suspending agent, sweetening agent, flavoring agent, aromatic agent, and preservative.

The examples of injectable drug for parenteral administration include sterile aqueous or non-aqueous solution, suspension or emulsion. The examples of aqueous solvent include, distilled water or saline solution for injection. The examples of nonaqueous solvent include, propylene glycol, polyethylene glycol or vegetable oils such as olive oil, alcohols such as ethanol, or polysorbate 80 (pharmacopeia name). Such composition may further contain isotonicity agent, preservative, wetting agent, emulsifier, dispersant, stabilizer, or solubilizer. These are sterilized, for example, by filtration through a bacteria-retaining filter, the addition of a bactericidal agent, or irradiation. These can also be produced as sterile solid composition and dissolved or suspended in sterile water or sterile injectable solvent, prior to use.

The examples of topical products include ointments, hard plasters, creams, jellies, poultices, sprays, lotions, eye drops, eye ointments, etc. The examples of topical products also include commonly used ointment bases, lotion bases, aqueous or non-aqueous liquids, suspensions, emulsions, etc. The examples of ointment or lotion bases include polyethylene glycol, propylene glycol, white vaseline, white beeswax, polyoxyethylene hardened castor oil, glycerin monostearate, stearyl alcohol, cetyl alcohol, lauromacrogol, sesquioleinic acid sorbitan, etc.

Transmucosal agents, such as inhalants and nasal spray, may be used in solid, liquid, or semi-solid form and can be manufactured according to conventionally known methods. For example, known excipients, pH adjusters, preservatives, surfactants, lubricants, stabilizers and thickeners may be added appropriately. Any suitable devices for inhalation or air delivery may be used for administration. For example, transmucosal agents may be administered using known devices such as metered dose inhalation devices or atomizers, as a compound alone or as a powder in a formulated mixture, or as a solution or suspension in combination with a pharmaceutically acceptable carrier. Dry powder inhalers and the like may be used for single or multiple doses, and dry powder or powder-containing capsules may be used. Alternatively, transmucosal agents may be administered in the form of a pressurized aerosol spray using suitable ejection agents, such as preferred gases, for example, chlorofluoroalkane, hydrofluoroalkane, or carbon dioxide, etc.

Dosage varies depending on the type of disease, symptom, age, and gender of patients. The usual dosage for oral administration is approximately 0.001 mg/kg-500 mg/kg per day for adults, which is administered once or divided into two to four doses. When administered by injection, injection is administered as a rapid intravenous infusion or intravenous drip at a dose of approximately 0.0001 mg/kg-10 mg/kg once or twice per day for adults. When administered by inhalation, a single or multiple doses of approximately 0.0001 mg/kg-10 mg/kg per adult per day is administered. When administered by transdermal drug, transdermal drug is applied once or twice per day at a dose of approximately 0.01 mg/kg-10 mg/kg per day for adults.

Compound A or compound B, or salts thereof, may be used in combination with various therapeutic or prophylactic agents for diseases for which compound A or compound B, or salts thereof is considered to be effective. The combination use may be simultaneous, or separate and consecutive, or at desired time intervals. The simultaneous administration formulation may be a compounding agent or a separate formulation.

### <<Method for Treating Mitochondrial Hypofunction Disease>>

The compound A or compound B may be used for the treatment of mitochondrial hypofunction disease. That is to say, this specification discloses a method for treating mitochondrial hypofunction disease comprising the step of administering a therapeutically effective amount of a compound represented by the formula (1) or a salt thereof, or a compound represented by the formula (2) or (3) or a salt thereof, to a patient having mitochondrial hypofunction disease patient.

### <<Compound a or Compound B for Use in the Treatment of Mitochondrial Hypofunction Disease>>

The compound A or compound B may be used for the treatment of mitochondrial hypofunction disease. That is to say, this specification discloses a compound represented by the formula (1) or a salt thereof, or a compound represented by the formula (2) or (3) or a salt thereof for use in the treatment of mitochondrial hypofunction disease.

### <<Use of Compound A or Compound B in the Manufacture of Pharmaceutical Composition>>

The compound A or compound B may be used for the manufacture of a pharmaceutical composition for the treatment or prevention of mitochondrial hypofunction disease. That is to say, this specification discloses use of a compound represented by the formula (1) or a salt thereof, or a compound represented by the formula (2) or (3) or a salt thereof, in the manufacture of a pharmaceutical composition for the treatment or prevention of mitochondrial hypofunction disease.

### <<Function>>

The mechanism by which compound A or compound B is effective in the treatment of mitochondrial hypofunction disease has not been analyzed in detail, but can be estimated as follows. Compounds A and B are Syk inhibitors, and it is presumed that the inhibition of Syk directly or indirectly promotes the formation of mitochondrial respiratory chain supercomplexes. It is presumed to be effective in the treatment or prevention of mitochondrial hypofunctional diseases by improving muscle function due to the formation of mitochondrial respiratory chain supercomplexes.

### EXAMPLES

The present invention now will be further illustrated by, but is by no means limited to, the following Examples.

### Example 1

In this example, the subunits constituting complexes I, III, and IV were fluorescently labeled.

In the respiratory chain complex I, NADH-ubiquinone oxidoreductase subunit B8 (NDUFB8) was labeled. In the respiratory chain complex III, Ubiquinol-cytochrome c reductase, 6.4 kDa subunit (UQCR11) was labeled. In the respiratory chain complex IV, Cytochrome c oxidase subunit 8A (COX8A) was labeled. In addition, ATP synthase F1 subunit gamma (ATP5F1c), which is not involved in the respiratory chain supercomplex, was also labeled as a control. The expression vectors for UQCR11, COX8A, and ATP5F1c were provided by the Osawa Laboratory of the Tokyo Metropolitan Institute for Geriatric and Gerontology. NDUFB8 was cloned from the cDNA of human breast cancer cell line MCF-7 using the following primers.

NDUFB8 Forward:
(SEQ ID No: 7)
5′-CCCGAGCTCGCCATGGGCGCGGTGGCCAGGGCC-3′
NDUFB8 Reverse:
(SEQ ID No: 8)
5′-GGGGTACCCCGATCTCATAGTGAACCACCCGC-3′

NDUFB8 (complex I), and UQCR11 (complex III) were fluorescently labeled by AcGFP. COX8A (complex IV) and ATP5F1c were fluorescently labeled by DsRed monomer. The mechanism of FRET generation by AcGFP and DsRed monomer is shown in FIG. 1C.

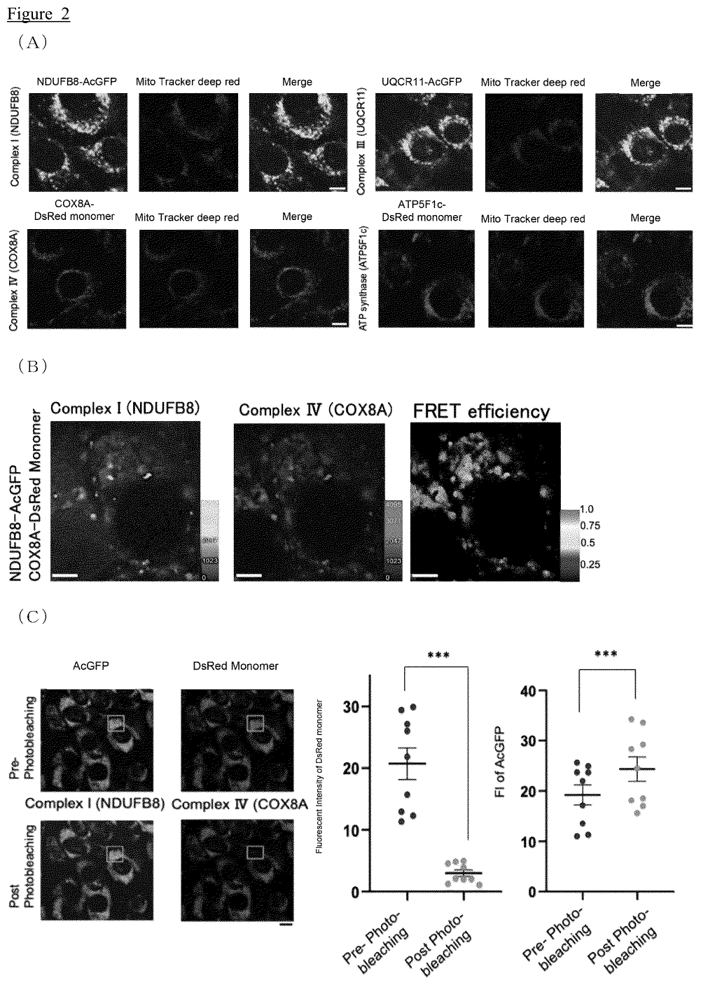

Each of the constructed plasmid vectors was transfected into C2C12 cell line using FuGENE HD (Promega, Madison, WI, USA). Transfected cells were examined by fluorescence microscopy for expression and localization. Fluorescences of AcGFP and DsRed monomer were observed in the transfected cells, and their localization in the cells were co-localized with Mito Tracker (FIG. 2A). Therefore, it was considered that the desired fusion protein was correctly expressed.

In addition, the protein in which the subunit NDUFB8 of respiratory chain complex I was labeled with AcGFP, and the protein in which the subunit COX8A of respiratory chain complex IV labeled with DsRed monomer, were co-expressed, whereby the fluorescence of DsRed monomer was detected at the excitation wavelength of AcGFP, and the FRET efficiency can be calculated (FIG. 2B). Therefore, it is considered that the proximity of the above fusion proteins can be detected as a FRET signal.

In addition, Acceptor Photobleaching was performed to verify whether the signals detected above reflect the FRET phenomenon. Nine ROI (regions of interest) were set in the image and the fluorescence intensities were recorded. These ROIs were irradiated by the excitation wavelength (558 nm) of DsRed monomer at for 3 minutes at maximum power. Immediately after the irradiation, a fluorescence image (Em:493-553 nm) of AcGFP excited at the excitation wavelength of AcGFP (488 nm) and a fluorescence image (Em:566-610 nm) of DsRed monomer excited at the excitation wavelength of DsRed monomer (558 nm) (Em: 566-610 nm) were obtained again. They were saved as the images after photobleaching, and the fluorescence intensity of the ROI was measured and recorded. The fluorescence intensity of DsRed monomer in the ROI was remarkably decreased after photobleaching compared to that before photobleaching, while that of AcGFP was increased (FIG. 2C). Thus, it was confirmed that the FRET phenomenon occurred between the above fluorescent fusion proteins.

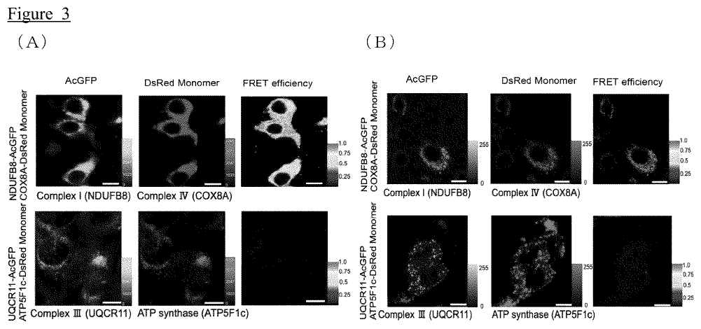

FRET signals between respiratory chain complex subunits in stably expressing cells were examined using C2C12 cells stably expressing both NDUFB8-AcGFP and COX8A-DsRed monomer and C2C12 cells stably expressing both UQCR11-AcGFP and ATP5F1c-DsRed monomer. In both cases of cells fixed with 4% paraformaldehyde (FIG. 3A) and live cells (FIG. 3B), signals at the acceptor emission wavelength were detected upon donor excitation by the combination of the subunit NDUFB8 of respiratory chain complex I and COX8A of respiratory chain complex IV. On the other hand, in the combination of the subunit UQCR11 of respiratory chain complex III and the subunit ATP5F1c of ATP synthase, no signal at the acceptor emission wavelength was detected upon donor excitation. This indicates that the detection of fluorescence of the DsRed monomer upon excitation of AcGFP reflects the formation of mitochondrial respiratory chain supercomplexes

### Example 2

In this example, it was measured whether the FRET signal obtained in Example 1 is altered by inhibiting the expression of COX7RP, which is known as a factor promoting the formation of respiratory chain supercomplexes.

When C2C12 cells were cultured in 6-well plates or 96-well plates, two types of siRNAs (#1 and #2) and two types of negative controls (siControl #1 and #2) were reverse transfected thereto at a concentration of 10 nM using Lipofectamine RNAiMAX transfection reagent (Invitrogen). The sequences of siRNAs are as follows.

siCox7rp #1
Sense:
(SEQ ID No: 9)
5′-CUGUGGCUUUACGUUAUGAUU-3′
Anti-sense:
(SEQ ID No: 10)
5′-UCAUAACGUAAAGCCACAGCA-3′
siCox7rp #2
Sense:
(SEQ ID No: 11)
5′-GGCUUUACGUUAUGAUUGACC-3′
Anti-sense:
(SEQ ID No: 12)
5′-UCAAUCAUAACGUAAAGCCAC-3′
siControl #1
Sense:
(SEQ ID No: 13)
5′-GUGGAUUUCGAGUCGUCUUAA-3′
Anti-sense:
(SEQ ID NO: 14)
5′-AAGACGACUCGAAAUCCACAU-3′
siControl #2
Sense:
(SEQ ID No: 15)
5′-GUACCGCACGUCAUUCGUAUC-3′
Anti-sense:
(SEQ ID No: 16)
5′-UACGAAUGACGUGCGGUACGU-3′

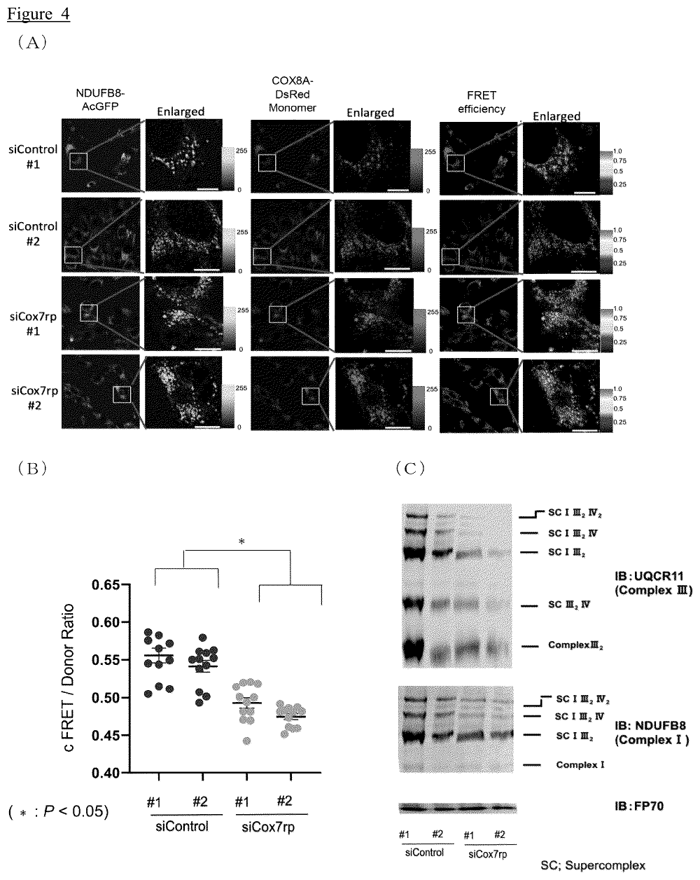

By using siRNA that suppresses the expression of Cox7rp, the FRET signal upon suppression of COX7RP expression in stably expressing cells in which NDUFB8-AcGFP and COX8A-DsRed monomer are co-expressed, was evaluated. FRET efficiency was decreased in siCox7rp treatment, compared to siControl (FIG. 4A).

In addition, Corrected FRET (cFRET)/Donor, which represents the FRET efficiency per molecule, was significantly decreased in siCox7rp treatment, compared to siControl treatment (FIG. 4B). The formation of respiratory chain supercomplexes was compared using the same siRNA and mitochondrial fractions extracted from C2C12 cells in which Cox7rp expression was suppressed, according to BN-PAGE. The formation of respiratory chain supercomplexes I+III2+IVn (n:1-2), I+III2, and III2+IV was decreased in siCox7rp treatment, compared to siControl treatment (FIG. 4C). These results indicate that the FRET signal reflects the formation of respiratory chain supercomplexes.

### Example 3

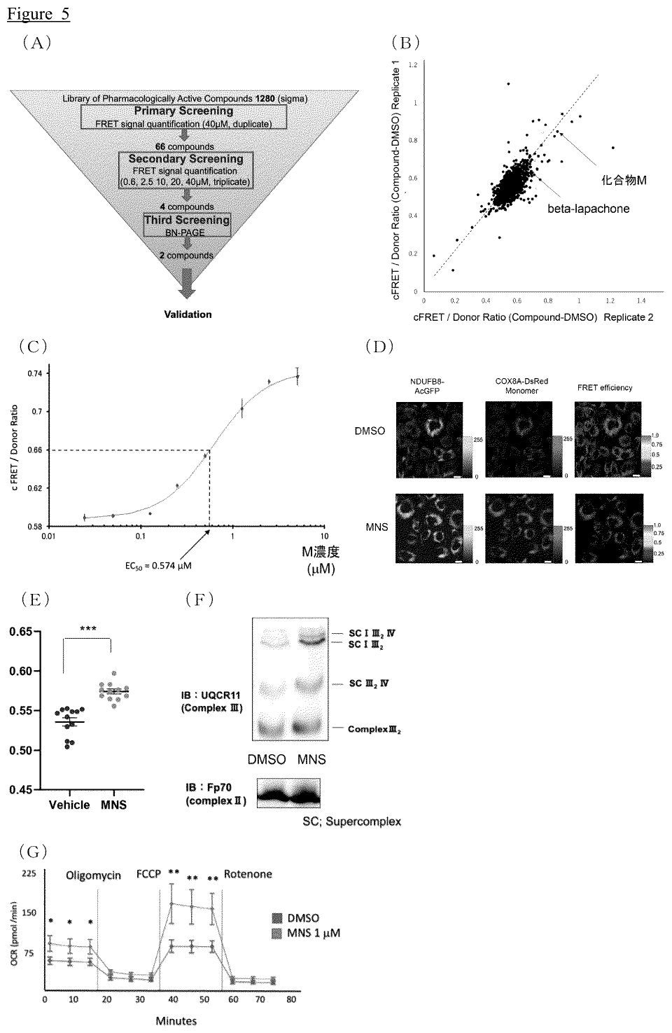

In this example, a three-step screening was performed by combining FRET and BN-PAGE of living cells from 1280 compounds in the Library of Pharmacologically Active Compounds (LOPAC 1280), in order to identify compounds that promote respiratory chain supercomplex formation (FIG. 5A).

In the primary screening, the cFRET/Donor of living cells with 40 μM of each 1280 compound was measured using the C2C12 expressing cells stably co-expressing NDUFB8-AcGFP and COX8A-DsRed monomer established in this study (FIG. 5B). In the secondary screening, cFRET/Donor was measured by adding 40, 20, 10, 2.5, or 0.6 μM of each compound. Mitochondrial fractions extracted from C2C12 cells treated with each of the four compounds, which showed increased cFRET/Donor even at low concentrations compared to the solvent condition, were compared in terms of respiratory chain supercomplex formation by BN-PAGE. The Syk/Src inhibitor, MNS (3,4-methylenedioxy-o-nitrostyrene) and the natural compound Beta-lapachone were screened in this screening. Beta-lapachone has already been reported to improve mitochondrial function.

MNS was added to stably expressed C2C12 cells stepwise from low to high concentrations, and the cFRET/Donor was measured. Based on these values, a dose-response curve was generated, and the 50% effective concentration (EC50) was estimated to be 0.574 μM (FIG. 5C). 0.574 μM is the concentration at which the Syk inhibitor is effective. In the following experiments, the respiratory chain supercomplex was examined in C2C12 cells under the condition of 1 μM addition.

Regarding MNS, a qualitative evaluation of the FRET signal was performed using stably expressed C2C12 cells in which NDUFB8-AcGFP and COX8A-DsRed monomer were co-expressed, obtained in Example 1. The FRET efficiency between the subunit fluorescent fusion proteins of respiratory chain complexes I and IV was increased under the MNS additive condition, compared to the DMSO additive condition as a solvent (FIG. 5D). The corrected value of cFRET/Donor was also significantly increased under the MNS additive condition than under the DMSO additive condition (FIG. 5E). Further, the formation of respiratory chain supercomplexes by BN-PAGE were compared using mitochondrial fractions extracted from C2C12 cells. The formation of respiratory chain supercomplexes I+III2+IV, I+III2, and III2+IV was increased under the MNS additive condition, compared to the DMSO additive condition (FIG. 5F). The oxygen consumption was measured using the XFp Extracellular Flux Analyzer, when MNS was added to C2C12 cells. Mitochondria of cells under adding 1 μM MNS showed significant increases in basal respiration and maximal respiration (spare respiratory capacity) (FIG. 5G), compared to the solvent-only control.

Oxygen consumption rate (OCR) was measured as follows. The C2C12 cells were seeded (5×10³ cells/well) in 6-well plates (Agilent Tech, CA, USA) and incubated overnight before measuring OCR and ECAR. One hour before the measurement, the medium was replaced with XF base medium containing 1 mM sodium pyruvate, 2 mM glutamate and 25 mM glucose (Agilent Tech). Basal respiration, ATP production, and maximal respiration (spare respiratory capacity) of C2C12 cells were measured using XFp Extracellular Flux Analyzer (Agilent Tech) and Mito Stress Kit for XFp (Agilent Tech). The drugs were added in the following order: 1.0 μM oligomycin, 0.5 μM carbonyl cyanide-p-trifluoromethoxyphenylhydrazone, and 0.5 μM rotenone. The baseline (basal) OCR was measured three times before and after the addition of the drugs.

### Example 4

In this example, the effects of Syk inhibitors, BAY61-3606 and GSK143, on the formation of supercomplexes and oxygen consumption were examined.

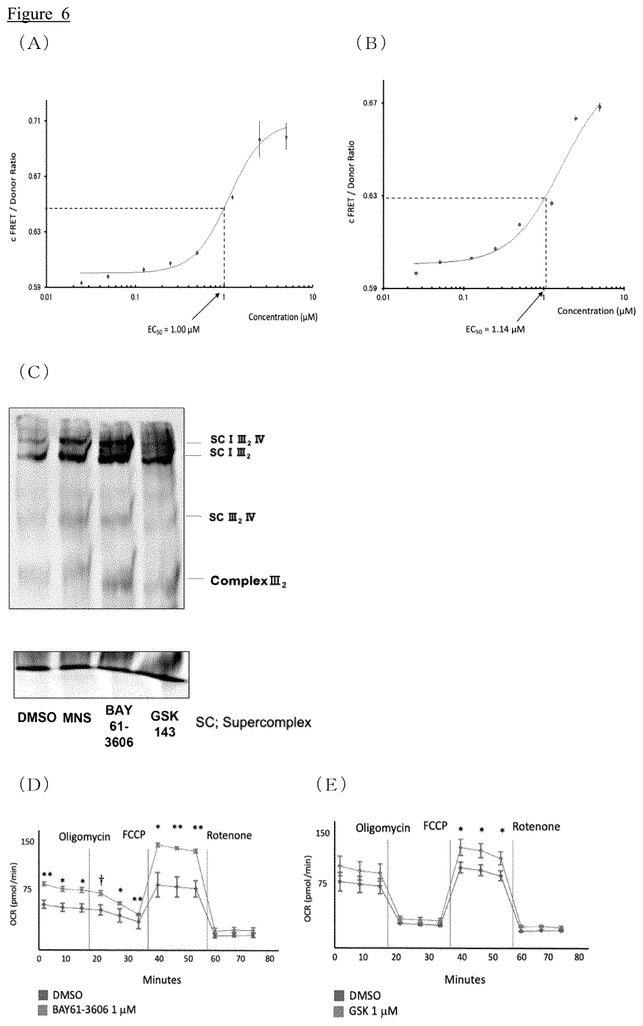

BAY61-3606 and GSK143 were added to stably expressed C2C12 cells stepwise from low to high concentrations, and the corrected value of cFRET/Donor was measured Based on these values, dose-response curves were generated, and the 50% effective concentration (EC50) were estimated to be 1.00 μM and 1.14 μM, respectively (FIGS. 6A and 6B). The formation of respiratory chain supercomplexes by BN-PAGE were compared using mitochondrial fractions extracted from C2C12 cells. As in the case of MNS, the formation of respiratory chain supercomplexes I+III2+IV, I+III2, and III2+IV was increased under the additive conditions of BAY61-3606 and GSK143, compared to those under the DMSO additive condition (FIG. 6C). The oxygen consumption was measured using the XFp Extracellular Flux Analyzer, when BAY61-3606 or GSK143 was added to C2C12 cells. Mitochondria of cells under adding 1 μM BAY61-3606 showed significant increases in basal respiration, ATP production, and maximal respiration (spare respiratory capacity) (FIG. D), compared to the solvent-only adding control. In mitochondria of cells under adding 1 μM of GSK143 condition, only maximal respiration (spare respiratory capacity) was predominantly increased (FIG. 6E).

### Example 5

In this example, the effects of Syk inhibitor, siRNA, on the formation of supercomplexes and oxygen consumption were examined. The base sequences of the used siRNAs are as follows

#1:
(SEQ ID No: 1)
5′-AAUGAAUUCAACAUACAGGGA-3′
(SEQ ID No: 2)
5′-CCUGUAUGUUGAAUUCAUUGA-3′
#2:
(SEQ ID No: 3)
5′-UUAAUCUUGACAGUAAGACAC-3′
(SEQ ID NO: 4)
5′-GUCUUACUGUCAAGAUUAAUU-3′

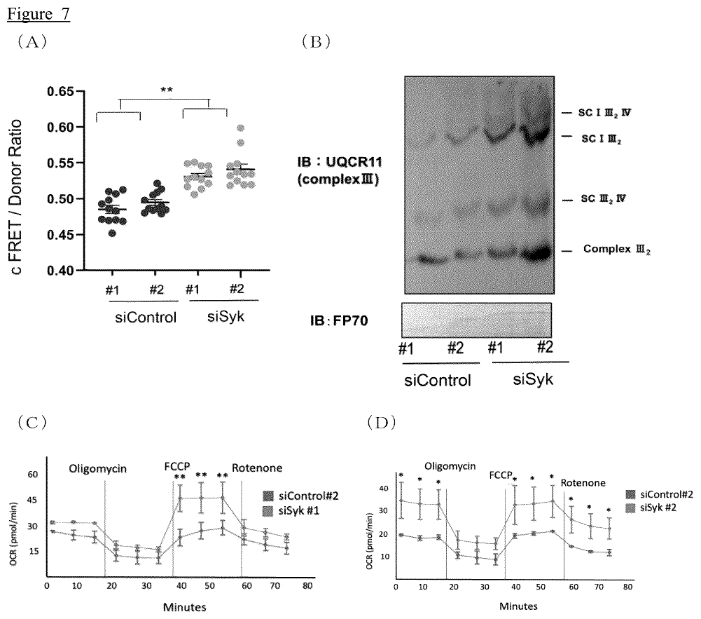

Both mRNA and protein expression levels of Syk in C2C12 cells were suppressed by the above #1 and #2 siRNAs. The above siSyk was transfected into cells stably expressing NDUFB8-AcGFP and COX8A-DsRed monomer, and FRET signals were examined. FRET efficiency was increased in siSyk treatment, compared to siControl treatment (FIG. 7A). The formation of respiratory chain supercomplexes was compared using the same siRNA and mitochondrial fractions extracted from C2C12 cells in which Syk expression was suppressed, according to BN-PAGE. The formation of respiratory chain supercomplexes I+III2+IVn (n:1-2), I+III2, and III2+IV was increased in siSyk treatment, compared to siControl (FIG. 7B). Oxygen consumption of C2C12 cells in siControl or siSyk treatment was measured using XFp Extracellular Flux Analyzer.

Mitochondria of cells treated with the two types of siSyk showed significant increases in basal respiration, maximal respiration (spare respiratory capacity), and ATP production in both siSyk treatments, compared to the siControl treatment (FIG. 7C, D).

### Example 6

In this example, MNS was administered to mice, to examine exercise tolerance.

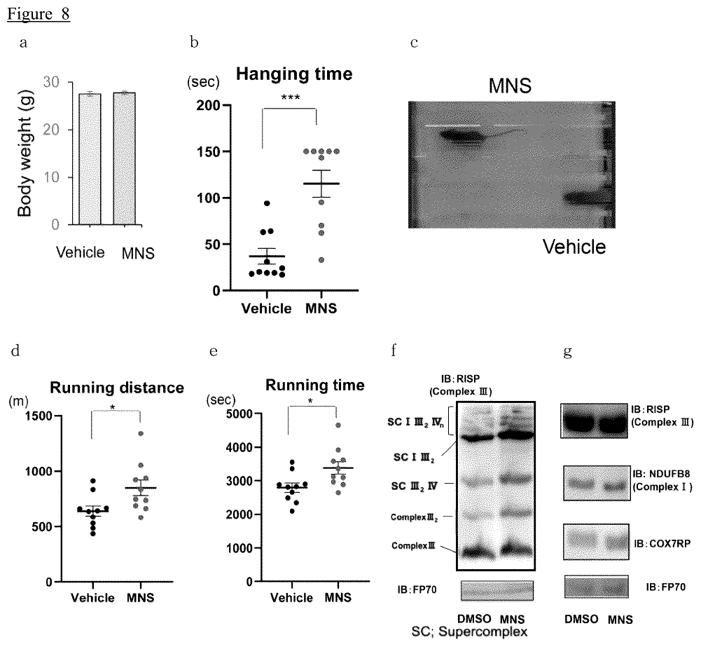

Seven-week-old DBA/2CrSc1 mice were injected intraperitoneally with MNS, or DMSO as a control. MNS administration caused no significant toxicity and no change in body weight (FIG. 8a). In the wire-hang test, the time holding the wire was significantly prolonged in the MNS-treated group, compared to the DMSO-treated group (FIG. 8b). In the treadmill test, the running distance and running time of the MNS-treated group were significantly longer than those of the DMSO-treated group (FIG. 8c-e). In addition, the formation of respiratory chain supercomplexes by BN-PAGE were compared using mitochondrial fractions extracted from the extensor digitorum longus and soleus muscles of the mice in each group. The formation of respiratory chain supercomplexes I+III2+IVn (n=1-2), I+III2, and III2+IV was increased in mitochondria of both extensor digitorum longus and soleus muscles under the MNS additive condition, compared to the DMSO additive condition (FIG. 8f). In this case, no significant difference was obtained between the expression levels of representative respiratory chain complex subunits and the protein expression level of COX7RP, a factor promoting the formation of respiratory chain supercomplexes (FIG. 8g).

### Example 7

In this example, BAY61-3606 or GSK143 was administered to mice, to examine exercise tolerance.

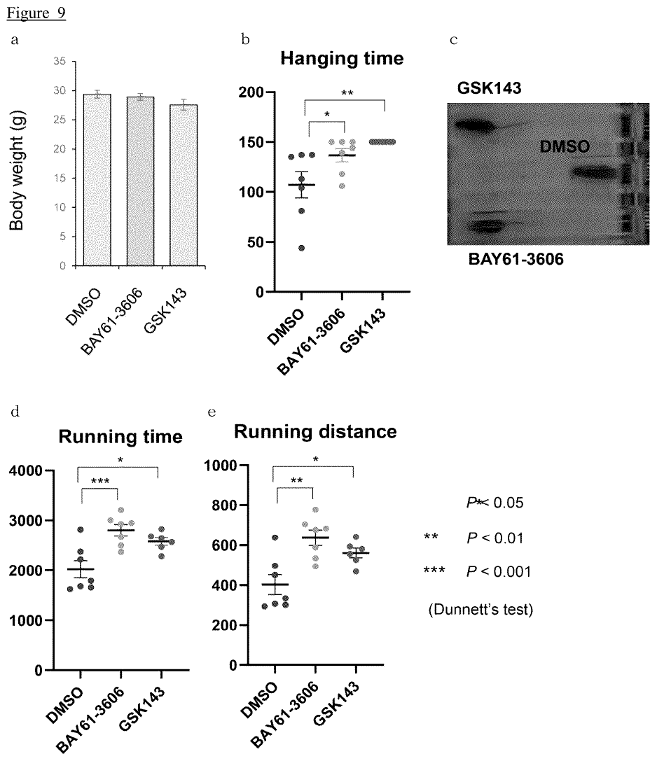

Seven-week-old DBA/2CrSc1 mice were injected intraperitoneally with BAY61-3606 or GSK143, or DMSO as a control. BAY61-3606 or GSK143 administration caused no significant toxicity and no change in body weight (FIG. 9a). In the wire-hang test, the time holding the wire was significantly prolonged in the BAY61-3606 or GSK143-treated group, compared to the DMSO-treated group (FIG. 9b). In the treadmill test, the running distance and running time of the BAY61-3606 or GSK143-treated group were significantly longer than those of the DMSO-treated group (FIG. 9c-e).

### INDUSTRIAL APPLICABILITY

According to the screening method of the present invention, it is possible to search for substances that can be used for the treatment of mitochondria-related diseases. Moreover, the agent for promoting formation of mitochondrial respiratory chain supercomplex of the present invention can be used to maintain or enhance muscle strength, or to treat muscle hypofunction disease or mitochondrial hypofunction disease.

1. An agent for promoting formation of mitochondrial respiratory chain supercomplex, comprising Syk inhibitor as an active ingredient.

2. The agent for promoting formation of mitochondrial respiratory chain supercomplex according to claim 1, wherein the Syk inhibitor is a double-stranded nucleic acid having an RNAi effect on a Syk gene.

3. The agent for promoting formation of mitochondrial respiratory chain supercomplex according to claim 2, wherein the double-stranded nucleic acid is siRNA of an oligonucleotide comprising the base sequences represented by SEQ ID Nos: 1 and 2, or siRNA of an oligonucleotide comprising the base sequences represented by SEQ ID Nos: 3 and 4.

4. The agent for promoting formation of mitochondrial respiratory chain supercomplex according to claim 1, wherein the Syk inhibitor is a compound represented by the following formula (1) or salt thereof: 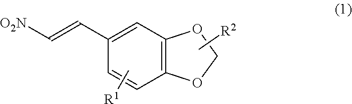
wherein R¹ and R² are independently a hydrogen atom, an alkyl group having 1 to 6 carbon atoms, or an alkoxy group having 1 to 6 carbon atoms, or
a compound represented by the following formula (2) or (3), or salt thereof: 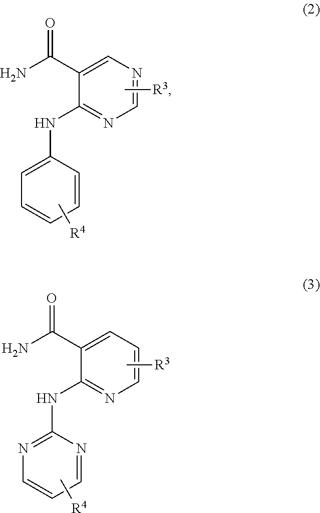
wherein, R³ is any of 1 to 4, which is independently a hydrogen atom, an alkyl group having 1 to 6 carbon atoms, an alkoxy group having 1 to 6 carbon atoms, an optionally substituted —NH-cycloalkyl group, an optionally substituted —NH-heterocycloalkyl group, an optionally substituted —NH-aryl group, or an optionally substituted —NH-alkylenaryl group, or two R³ taken together with two elements to which they are attached may form a 5-membered or 6-membered heterocyclic ring, and
R⁴ is any of 1 to 5, which is independently a hydrogen atom, an alkyl group having 1 to 6 carbon atoms, an alkoxy group having 1 to 6 carbon atoms, an aromatic heterocyclic group, or an optionally substituted aryl group, or two R⁴ taken together with two elements to which they are attached may form a 5-membered or 6-membered heterocyclic ring.

5. The agent for promoting formation of mitochondrial respiratory chain supercomplex according to claim 4, wherein the Syk inhibitor is 3,4-methylenedioxy-β-nitrostyrene represented by the following formula (4): 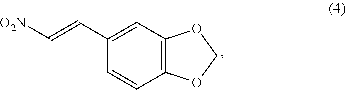 [SMILES: O=[N+]([O-])/C=C/c1ccc2c(c1)OCO2]
2-[[7-(3,4-dimethoxyphenyl)imidazo[1,2-c]pyrimidin-5-yl]amino]pyridine-3-carboxamide represented by the following formula (5): 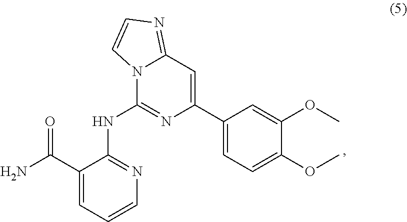 [SMILES: COc1ccc(-c2cc3nccn3c(Nc3ncccc3C(N)=O)n2)cc1OC]
or 2-[[(3R,4R)-3-aminotetrahydro-2H-pyran-4-yl]amino]-4-[(4-methylphenyl)amino]-5-pyrimidinecarboxamide represented by the following formula (6):

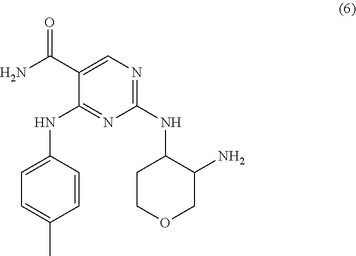 [SMILES: Cc1ccc(Nc2nc(NC3CCOCC3N)ncc2C(N)=O)cc1]

6. A composition for maintaining or enhancing muscle strength, comprising an agent for promoting formation of mitochondrial respiratory chain supercomplex according to claim 1.

7. A pharmaceutical composition for treating or preventing muscle hypofunction disease or mitochondrial hypofunction disease, comprising an agent for promoting formation of mitochondrial respiratory chain supercomplex according to claim 1.

8. A combination of:
a donor fusion gene in which a gene encoding a FRET donor is fused to a gene encoding at least one protein constituting mitochondrial respiratory chain complex I, a gene encoding at least one protein constituting mitochondrial respiratory chain complex III, a gene encoding at least one protein constituting mitochondrial respiratory chain complex IV, or a COX7RP gene, and
an acceptor fusion gene in which a gene encoding a FRET acceptor is fused to a gene other than the gene to which the gene encoding the FRET donor is fused (with the proviso that the genes to which the FRET donor and FRET acceptor bind are not genes that encode proteins constituting a single mitochondrial respiratory chain complex).

9. A combination of a vector comprising the donor fusion gene defined as in claim 8, and a vector comprising the acceptor fusion gene defined as in claim 8.

10. A cell comprising the combination of the fusion proteins according to claim 8.

11. A method for screening a substance involved in a formation of mitochondrial respiratory chain supercomplex, comprising the steps of:
contacting the cell according to claim 10 with a substance to be tested, and measuring a Förster resonance energy transfer.

12. A cell comprising the combination of the vectors according to claim 9.

13. A method for treating or preventing muscle hypofunction disease or mitochondrial hypofunction disease, comprising a step of administering to a subject in need thereof, the agent for promoting formation of mitochondrial respiratory chain supercomplex according to claim 1, in an amount effective thereof.


SEQUENCE LISTING (18 sequences)

SEQ ID NO: 1 - RNA, 21 nt, Artificial Sequence
  siSyk#1 sense
aaugaauucaacauacaggga

SEQ ID NO: 2 - RNA, 21 nt, Artificial Sequence
  siSyk#1 anti sense
ccuguauguugaauucauuga

SEQ ID NO: 3 - RNA, 21 nt, Artificial Sequence
  siSyk#2 sense
uuaaucuugacaguaagacac

SEQ ID NO: 4 - RNA, 21 nt, Artificial Sequence
  siSyk#2 antisense
gucuuacugucaagauuaauu

SEQ ID NO: 5 - RNA, 23 nt, Mus musculus
ucaaugaauucaacauacaggga

SEQ ID NO: 6 - RNA, 23 nt, Mus musculus
aauuaaucuugacaguaagacac

SEQ ID NO: 7 - DNA, 33 bp, Artificial Sequence
  NDUFB8 Forward
cccgagctcgccatgggcgcggtggccagggcc

SEQ ID NO: 8 - DNA, 32 bp, Artificial Sequence
  NDUFB8 Reverse
ggggtaccccgatctcatagtgaaccacccgc

SEQ ID NO: 9 - RNA, 21 nt, Artificial Sequence
  siCox7rp #1 sense
cuguggcuuuacguuaugauu

SEQ ID NO: 10 - RNA, 21 nt, Artificial Sequence
  siCox7rp #1 anti-sense
ucauaacguaaagccacagca

SEQ ID NO: 11 - RNA, 21 nt, Artificial Sequence
  siCox7rp #2 sense
ggcuuuacguuaugauugacc

SEQ ID NO: 12 - RNA, 21 nt, Artificial Sequence
  siCox7rp #2 anti-sense
ucaaucauaacguaaagccac

SEQ ID NO: 13 - RNA, 21 nt, Artificial Sequence
  siControl #1 sense
guggauuucgagucgucuuaa

SEQ ID NO: 14 - RNA, 21 nt, Artificial Sequence
  siControl #1 anti-sense
aagacgacucgaaauccacau

SEQ ID NO: 15 - RNA, 21 nt, Artificial Sequence
  siControl #2 sense
guaccgcacgucauucguauc

SEQ ID NO: 16 - RNA, 21 nt, Artificial Sequence
  siControl #2 anti-sense
uacgaaugacgugcgguacgu

SEQ ID NO: 17 - DNA, 4973 bp, Homo sapiens
accccggcggcggctggagagcgaggaggagcgggtggccccgcgctgcgcccgccctcgcctcacctggcgcaggtggacacctgcgcaggtgtgtgccctccggcccctgaagcatggccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccccgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcggatgatcgggatatgcgaggccgagtcctggatgctggttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattacgccaagatcagtgatttcggactttccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaactaaccgctcccgcacctgtcggtggctgcctttgatcacaggagcaatcacaggaaaatgtatccagaggaattgattgtcagccacctccctctgccagtcgggagagccaggcttggatggaacatgcccacaacttgtcacccaaagcctgtcccaggactcaccctccacaaagcaaaggcagtcccgggagaaaagacggatggcaggatccaaggggctagctggatttgtttgttttcttgtctgtgtgattttcatacaggttatttttacgatctgtttccaaatccctttcatgtctttccacttctctgggtcccggggtgcatttgttactcatcgggcccagggacattgcagagtggcctagagcactctcaccccaagcggccttttccaaatgcccaaggatgccttagcatgtgactcctgaagggaaggcaaaggcagaggaatttggctgcttctacggccatgagactgatccctggccactgaaaagctttcctgacaataaaaatgttttgaggctttaaaaagaaaatcaagtttgaccagtgcagtttctaagcatgtagccagttaaggaaagaaagaaagaaaaaaaaaaaaggcctggatactgcttttgctgtctctgttatgagatggaagacttacatgtttgtgataaaaggggaccatgagaatgaattggcttggcttactttccccctgaaatcctctctcctgcagactgtcttgaagacctggtgactggtaaataaagccctgcatggaggctgcacagcaggggcaagaggcccatcccccagcatctcactgaggacagcttcaggctgccttcctctgaacgtggtccacaccttcctctcctccacagagagggtgccgccagaatcccctgtcgctttctgtgtctgcaatggggggcagcacagggatcaaagccatctaaagagtttccaaagaaagtattaattcagaacaagccaaagaccctgagcctcaccacaaacaggccttttggagtgtgaatttgagttgaagatacaagatcggagaatgattttctggtcttaactaatcctcatcttcatgtttgatctttaagaagtcatcacccattgatttcagttttgctgtacctcttgaaagttaaagagacatctcagcactttaggaggccgaggcgggtggatcacttgaggtaaggagtttgagactagcctggccaatatggtaaaaccccatctctactaaaaatacaaaaattagccgggcatggtggcatgtgtctgtagtcccagctactcgggaggctgaggcaggagaatcgcttgaacccaggaaacggaggtcgcagtgagccaagatcatgccactgcactccagcttgggcatcacagcgagactctgtcaaaacaaacaaacaaaaaaacaacttaaagaggtaatttagccatcattcttatgccagcagatataaataaacttggacccatctggtcttcagctaaacctgagacattttaaagtgcatggacagccatggacagcaggccctcctctaacaggggatgcaaggcatggagaaagacaatcagtacccaagctcagccacagaagacaggagtcactcatataacttgtgtttagaagtttttggtagccacgcacactttctgaaatcacactatctggtggtttaatcatatttttaaagacagaatccctgagtgctgagcagattctcaaaacacatttagaatccctgaaattagaaagatcaatgacaaaatatctgtcagccaggccacaaacaggtgtaaaattatgaaaggagtggttggatgtgccaagtttggtaaagtggtgactgcatctgagaaagaggctgtgaggctgaactcttggtggcttccttctgtaacttccagagggagtcttcaacacaggccccgtgctcgtaggaatacggtagcacctatgtaggaagtgcgtggagttttctgtcttctttctgtgtgatttttggcctttttatcagcacttctcccctcccaggagcctggggatgccaaacatccagaatgtgatgggacaagatgggggcaggggcctcacctccctgcagaggtccggccaggtctccttgtccctggacaatctcctgagcctctctgcttggtggagcaggcacctgtgtgcagaattcccactgtggccagcacgaggaagtcttttctagtgaaaatgtgtcttgtggtcaggaataattatcctttcccctgtagccaccaaggagggcaaatagagaaaggtaacctaattgaaggattggtcatgtgaaaagggctacatttgggaagctgggaaaggcctccaggcttctagagcagctagcttgggctggattctcatacccaggctgccccttggattgttctacccaagcttttccctggggtctgggctcactccataaggtaaggtgccttttaccttatggtccttctttagcaggtaacaaaggagcatcaggggcaggctgccctggtggcatcacactggctagtgaggccgtgaatatcttgtcccccagcagggccgacagtttctatcacagaaaacagtgtgttcagtggtgaaaatcgttgcatgcatgttttcatctgagcgtgtccttctcccatactccctatcagccagccctgcctgtagctgctgtatggtgattgcacttggacatcagtccaatgactgcaagtcggcctggattttcacttgcagaggctacagctgcattgtcaggtctcccagccctgcagagagctccctccactggttagcagtgtgttgtgttttccattcatttcagaagagctacattgtgtcactggacatttttaaaaactgtgatttttaataaaaatttaaaatttg

SEQ ID NO: 18 - DNA, 5148 bp, Mus musculus
ccacttccctagttacagacaccacttaacagcaggaaacctccacttgctctcctctgccttctctccattgcagccattcccattttcaagactgccagtctggtcctttcaacgttccatgctgcctggtgcccgggagcccaggccttctgtgactccaggacaggaaggtacttctccatacacttcccagaactctgaaggggtgcagacatggcgggaagtgctgtggacagcgccaaccacctgacctacttttttggcaacatcacccgggaagaggctgaagactacctggtccagggaggcatgaccgatgggctctacctgctacgccagagccgcaattacctgggtggttttgctttgtcggtggctcacaacaggaaggcacaccactacaccatcgagagggaacttaatggcacctacgccatctccgggggcagggcccatgccagcccagcagacctctgccattaccactcccaggaacctgatggccttatctgcctccttaagaagcccttcaaccggcccccgggagtacagcccaagaccggaccctttgaggacctgaaggagaacctcatcagggaatatgtgaaacagacctggaaccttcagggccaggctctggagcaagccatcatcagccagaagccccagctggagaagctgatcgccaccacggcccatgagaagatgccctggttccatggcaacatctccagagatgaatcagagcagacggtcctcatagggtcaaagaccaatggaaaattcctgatcagggccagagacaacagcggctcctatgctctgtgcctgctgcacgaagggaaagtattgcactaccgcattgacagggacaagaccgggaagctctccattcctgaggggaagaagtttgacaccctctggcagctagtggaacattactcttacaagccagatgggctactaagagtcctcacggtaccatgccaaaagattggtgcacagatgggccacccaggaagcccaaatgcccatcccgtgacttggtcaccgggtggaataatctcaaggatcaaatcctactccttcccaaagcctggccacaaaaagcctgccccaccccaagggagccgtccagagagcactgtgtccttcaacccctatgagccaacgggagggccctggggcccagacagaggccttcagagagaagccctgcccatggacacagaggtgtacgagagcccttatgctgaccctgaagagatccggcccaaagaggtctacctggacaggagcctgctgaccctggaggacaatgaactgggctccggtaacttcgggactgtgaaaaagggatactaccaaatgaaaaaagttgtgaaaaccgtggctgtgaaaatcctgaagaacgaggccaacgacccggctttgaaggacgagctgctggcagaggcgaacgtcatgcagcagctggacaacccctacattgtgcgcatgatcggaatctgcgaggcggagtcctggatgctggtgatggagatggcggagctggggccgctcaacaagtacctgcagcagaacaggcacattaaggataagaacatcatagagctggttcaccaggtttccatggggatgaagtatttggaagagagcaactttgtgcacagagatctggctgcgcggaacgtgcttctggtcacacagcactatgccaagatcagcgatttcggtctttccaaagccctgcgtgctgatgaaaactactacaaggcccagacccacgggaagtggcccgtgaagtggtacgcccccgaatgcatcaactactacaagttctccagtaagagtgacgtctggagcttcggagtcctgatgtgggaagcgttctcctatgggcagaagccctacagagggatgaaagggagcgaagtgaccgccatgctggagaaaggagagcggatggggtgccctgcaggatgcccgagagagatgtacgacctgatgaacctgtgctggacttacgatgtggagaacaggccaggattcacagctgtggaactgaggcttcgcaattactactacgacgtggttaactaacagctccggcgcctgtccgtgcacaccacggattcccaagcgatcacaggaaattcattcagatgaactggctctcagagtttcatctccctctgcccggagtgagagctaagctaacataggactcaccctcacaacaggtctggtgcccagagacagacaagcagcaagaccttggggcctgtggagtcgtctctctggtctttgtttccatctgtgtggtcttcgctatcggtcacgtttgggagccatttccaaattctcttgacatcattccgttcctctgggtccaggatttcagtgtcccctgaagatcagaaaagaaaagtgctttgccactccagcaaaggacataaagagaaacctcgctggtgcagtcagcaagctggaaggtggttaaggacaatgacaagcctggacactggttttactctcagcgctgagacagagatactgtgtggatgacatgctgagatgtgctggcttggctgacacgcccctggagtccacctccctgcagtcgtgtgtgtttgctggaggaggtgtgaataacgttctgtgtggaggctgccgggggaggtgagctcccatgatcctctgcgactgagcagaagtgtgtcaggcaagggtcagactcccctctctgcagagggaatgcatccaggactccttgttgctgccactaaaaaattgccaaccagcaacttaagggaggaatggctcatttgagctcacagttggaggggataggccatcatggtggaaaagacaacaggcaggaaaggcttggtggcatgaaaaggaagctggaaggtcacatggcatccacattcaggaaacaaagaatgaacaggaagtggggccaggctataaggccctaaaggccacctccggtgacccagttccttcagatccatctatcaaagattccacaggcctctcagacactgaatgccaacagctggggaccaaatgttcaagcacacaaggtgcagtgccaggcaggggccttcctgaatgattacacagtgtggtatcctgacagagcgggtgagccatctcaggtctgtccccttatgagccccgtttaccatcccagattcccccttgggacccccctcatctggatcattcccctccccccaggccctatctccaaacacctaggtactaagtgaacctaggtactaagtcttcaacacatgaattctgaggggacacattcatactggagccgatggcagcctggtgaccaagaccatctcaggatgagtttctgaatggaatcttcattcgccataaaaccagagatgtgaccttccccacaaggaagccttgctaagtgcgacattgagctgaggacacacaagctgagaattccccctctgtcctcctagccttggctaactactgaccatacaccgaatgtcacccattcacggcacttttgctgaggacctaaaagctacaaaggctgcatagccttccatctaggtctagggacatgtacagatgggagcatctcaccttcacctctgagacactgggacattcacagccagggatggtacaggagtccctgctggtggtgggctcaatgaacagagacgatggtgaaatgggtgcctctctttagaccagtggttctcaaccttcctaatgctgagatctttcaatacaggtccttgcgttgtggcgactcccaaccatgaagttattttgttgctacattgtcactgtaattttgctactatgatgaatcgtagtgtagatatctgatatgcaggatatcggatatgtaaatatctggtatgcaggatatgtgaaatgagggcacatcccacaggttgggaaccactgcctttgatagggaggcactatgcttagagcatggttaggaagtatttagaagctattggtgtgtcttggaattgtgttagtaggtcaaccgtcctgtggagggaaggaagggggtggaggaaatccctgtatgttgaattcattgagcagacctgcagaacacagccttggtgaggaagtcccatgggtcagaccacctgtagatttagtcaggagaggaagggccactgtatagttatggagacaggactgtctgctctgagcaagaagttctctgctgagctcctagcctaccttcccttgtccccagggctatagaaaggccacctcgaagaccagggagcatgtgtgagaagtctgtggaggcctctgccttcttcctggcctctctagcagtgcctctccttcccagggatttgggactgaccgttatctggctatgactgagcagagtggagggatgggctttgccccccttcagaaagccgaaaccggttcccatgtgcagagtcacatgtggccatcgaccagggctctcatctggtgaaaactgtgtcttactgtcaagattaattctctcccctgggccactgaggagggcagatggcaagaggatcctccctagaggatttagactatgaatgcccactaaatttgcaaggtcagaaactagccaaggtccttctcaggcatctatccttaacttggtctctccacaagctgcctctgatgctccgagccccttctgcctggcagctgggctccctgtgtgcttttacctatgacccttggtcagcagagtacaaggagcacctaagctgcctcgccacctcccactgactgcaaacaccacatacctcatgcggaaccgaatgtctctccagtaaacaccccagccagccattcataaaaacctgtctctgtgtgcgctgggacatgtcctccctgtaccccgggcctgctttgtgtgccagacagtgactccacagggatgccaggtctgtgattgcattgctccttgcagagggaagccctcggttgtccttctctgggaatgctgccacccagagcagcacacagtgattccctgttatttcagaaattattttaaataaagatctactattagtcttgtgaaaaaaaaaaaaaaaaaaaaaaaaaaaaaaaaaaaaaaaaaaaaaa